(19) United States
(12) Patent Application Publication
(10) Pub. No.: US 2022/0354811 A1
(43) Pub. Date: Nov. 10, 2022

(54) METHODS AND COMPOSITIONS FOR MODULATING MACROPHAGES POLARIZATION

(71) Applicants: INSERM (INSTITUT NATIONAL DE LA SANTÉ ET DE LA RECHERCHE MÉDICALE), Paris (FR); UNIVERSITE COTE D'AZUR, Nice (FR)

(72) Inventors: Arnaud JACQUEL, NICE (FR); Guillaume ROBERT, NICE (FR); Patrick AUBERGER, NICE (FR); Paul CHAINTREUIL, NICE (FR)

(21) Appl. No.: 17/764,583

(22) PCT Filed: Oct. 2, 2020
(86) PCT No.: PCT/EP2020/077670

§ 371 Date: Mar. 29, 2022

(30) Foreign Application Priority Data
Oct. 3, 2019 (EP) 19306276.7

(51) Int. Cl. A61K 31/197; A61P 11/00; A61K 45/06

(52) U.S. Cl. CPC A61K 31/197; A61P 11/00; A61K 45/06

(57) ABSTRACT

Inventors have surprisingly found that Emricasan is a much more potent inhibitor of monocyte differentiation compared to q-VD-OH by its ability to efficiently inhibit caspase-8, which is instrumental to this process. In addition, they have demonstrated that Emricasan alleviates the IL4-mediated M2-like polarization of human macrophages. Moreover, Emricasan also hampers bleomycin-induced pulmonary fibrosis in mice, a disease associated with an infiltration of M2-macrophages. Finally, caspase-8 deficient mice were found to be resistant to bleomycin-induced pulmonary fibrosis. As a whole, their findings indicate that the beneficial effect of Emricasan relies on its ability to inhibit caspase-8, and its capacity to prevent monocyte differentiation and M2 polarization of macrophages. Accordingly, the invention relates to a caspase 8 inhibitor for use in the polarization of macrophages.

### FIELD OF THE INVENTION

The invention relates to methods and compositions for modulation of macrophages. More particularly, the invention relates to treat cancers and fibrosis by modulating macrophages polarization.

### BACKGROUND OF THE INVENTION

Caspases (cysteine-aspartic proteases) are cysteine proteolytic enzymes whose functions are inextricably linked with the process of programmed cell death in all metazoans. Cell death is a fundamental process that maintains tissue homeostasis, remove unwanted or damaged cells and ensures recycling of cellular constituents promoting further growth. To date, 12 caspases are referenced in human and are known for driving cell death through apoptosis, pyroptosis, or necroptosis. Caspases are synthetized as inactive zymogens and predominantly cleave, once activated, their substrates on the C-terminal side of an aspartate residue, less frequently after glutamate and in rare cases following phosphoserine residues. The set of proteomic approaches allowed to highlight over 1500 caspases substrates and delivered a much clearer blueprint of caspase targets and caspase specificity. The consequence of the cleavage on the function of most substrate proteins remains to be elucidated.

Beyond their originally described role as conveyors of programmed cell death, caspases are involved in some non-apoptotic functions including proliferation, inflammation and cell differentiation. In this context, we and other teams have shown that the differentiation of human blood monocytes into M2-like macrophages, i.e. anti-inflammatory macrophages, is a caspase-dependent process. Monocytes are circulating blood leukocytes that play important role in tissue homeostasis and in the regulation of inflammatory response. They have the property to migrate into tissues where they differentiate into morphological and functionally heterogeneous cells, including macrophages. The differentiation of peripheral blood monocytes into M2-like macrophages can be elicited by colony-stimulating factor-1 (CSF-1). The biologic effects of CSF-1 are mediated through the CSF-1 receptor (CSF-1R) that triggers activation of the PI3K-AKT and AMPK pathways, which are implicated in the respective activation of caspases and autophagy, two key processes required for CSF-1-induced macrophage differentiation. Our previous studies have established that physiological monocyte differentiation triggered by CSF-1R engagement is dependent on the kinase AKT, which induces the formation of a multi-molecular complex composed of the adaptor Fas-associated death domain (FADD), the serine-threonine kinase RIP1, FLIP and procaspase-8. Caspase-8 activation within this complex triggers a limited activation of effector caspases that cleave specific intracellular proteins. The contribution of these cleavages to the CSF-1—driven monocyte-to-macrophage differentiation remains poorly understood.

### SUMMARY OF THE INVENTION

The present invention relates to a caspase 8 inhibitor for use in the polarization of macrophages. In particular, the present invention is defined by the claims.

### DETAILED DESCRIPTION OF THE INVENTION

Inventors have surprisingly found that Emricasan is a much more potent inhibitor of monocyte differentiation compared to q-VD-OPh by its ability to efficiently inhibit caspase-8, which is instrumental to this process. In addition, they have demonstrated that Emricasan alleviates the IL4-mediated M2-like polarization of human macrophages. Moreover, Emricasan also hampers bleomycin-induced pulmonary fibrosis in mice, a disease associated with an infiltration of M2-macrophages. Finally, caspase-8 deficient mice were found to be resistant to bleomycin-induced pulmonary fibrosis. As a whole, their findings indicate that the beneficial effect of Emricasan relies on its ability to inhibit caspase-8, and its capacity to prevent monocyte differentiation and M2 polarization of macrophages.

Here, the inventors show that Emricasan is an efficient inhibitor of caspase-8 activity in primary human monocytes exposed to CSF-1, which modulates the response of monocyte-derived cells to the cytokine IL-4. As monocytes and monocyte-derived cells are major actors of tissue fibrosis development and CSF-1R inhibitors could prevent radiation-induced lung fibrosis, the inventors tested the ability of Emricasan to be an alternative to CSF-1 and CSF-1R targeting inhibitor in reducing lung fibrosis development in bleomycin-treated mice. A similar prevention of lung fibrosis development was observed by deleting caspase-8 in mouse granulo-monocytes. Altogether, these observations position Emricasan as an alternative to CSF1R inhibitors to modulate monocyte functions in human diseases.

This new finding with either Emricasan alone or in combination with other therapeutics seem to be very promising in patients with macrophages related diseases such as cancer and fibrosis.

### Method for Macrophages Polarization

Accordingly, in a first aspect, the present invention relates to a caspase 8 inhibitor for use in the polarization of macrophages.

In a particular embodiment, the caspase 8 inhibitor for use according to the invention inhibits the polarization of macrophages type 2.

In a particular embodiment, the caspase 8 inhibitor for use according to the invention activates the polarization of macrophages type 1.

As used herein, the term “macrophages” refers to cells that have the highest plasticity of the hematopoietic system. They derived from monocyte precursors undergo specific differentiation depending on the local tissue environment. The various macrophage functions are linked to the type of receptor interaction on the macrophage and the presence of cytokines. Two distinct states of polarized activation for macrophages have been defined: the classically activated (M1) macrophage phenotype and the alternatively activated (M2) macrophage phenotype. Similar to T cells, there are some activating macrophages and some suppressive macrophages, therefore, macrophages should be defined based on their specific functional activities. Granulocyte macrophage colony stimulating factor (GM-CSF) and macrophage colony stimulating factor (M-CSF) are involved in the differentiation of monocytes to macrophages. Human GM-CSF can polarize monocytes towards the M1 macrophage subtype with a “proinflammatory” cytokine profile (e.g. TNF-alpha, IL-1beta, IL-6, IL-12 and IL-23), and treatment with M-CSF produces an “anti-inflammatory” cytokine (e.g. IL-10, TGF-beta and IL-1ra) profile similar to M2 macrophages. Classically activated (M1) macrophages have the role of effector cells in TH1 cellular immune responses. The alternatively activated (M2) macrophages appear to be involved in immunosuppression and tissue repair.

As used herein, the term “polarization” refers to the phenotypic features and the functional features of the macrophages. The phenotype can be defined through the surface markers expressed by the macrophages. The functionality, can be defined for example based on the nature and the quantity of chemokines and/or cytokines expressed, in particular secreted, by the macrophages. Indeed, the macrophages present different phenotypic and functional features depending of their state, either pro-inflammatory M1-type macrophage or anti-inflammatory M2-type macrophage. M2-type macrophages can be characterized by the expression of surface markers such as CD206, CD163, PD-L1 and CD200R and then secretion of cytokines such as CCL17, IL-10, TGFb. M1-type macrophages can be defined by the expression of surface markers such as CD86 and CCR7 and the secretion of cytokines such as IL-6, TNF-a and IL12p40. In the context of the invention, caspase 8 inhibitor allows to modulate the polarization of macrophages population by inhibiting the M2-type macrophages and/or favoring the M1 -type macrophages.

As used herein, the term “macrophages type 1” known as classically activated macrophages (M1 macrophages or TAM-M1), refers to cells activated by lipopolysaccharides (LPS) or by double signals from interferon (IFN)-γ and tumor necrosis factor-α (TNF-α). This first type of macrophage are able to kill microorganisms and tumor cells.

As used herein, the term “macrophages type 2” also known as “immunosuppressive tumor-associated macrophages M2” or “M2 macrophages or Tumor-associated macrophages type M2 (TAM-M2)” refers to a type of blood-borne phagocytes, derived from circulating monocytes or resident tissue macrophages. Exposure to IL-4, IL-13, vitamin D3, glucocorticoids or transforming growth factor-β (TGF-β) decreases macrophage antigen-presenting capability and up-regulates the expression of macrophage mannose receptors (MMR, also known as CD206), scavenger receptors (SR-A, also known as CD204), dectin-1 and DC-SIGN.9 M2-polarized macrophages exhibit an IL-12^{low}, IL-23^{low}, IL-10^{high} phenotype. This second type of macrophage plays an important role in stroma formation, tissue repair, tumor growth, angiogenesis and immunosuppression. In blood cancers, TAMs are the most abundant inflammatory cells and are typically M2-polarized with suppressive capacity (1) that stems from their enzymatic activities and production of anti-inflammatory cytokines, such as TGFβ (Fuxe et al., Semin Cancer Biol, 2012, 22:455-461). High TAM levels have been associated with poorer BC outcomes (Zhao et al., Oncotarget, 2017, 8:30576-86. Therefore, several strategies are currently under investigation, such as the suppression of TAM recruitment, their depletion, or the switch from the pro-tumor M2 to the anti-tumor M1 phenotype in patients with TNBC (Georgoudaki et al., Cell Reports, 2016, 15:2000-11).

As used herein, the term “caspase 8” refers to cysteine-dependent aspartate-directed proteases. Caspases are a family of cytosolic aspartate-specific cysteine proteases involved in the initiation and execution of apoptosis. Caspase-8 is a cysteine protease known for its roles in Fas-induced apoptosis and lymphocyte activation. Activation of caspase-8 is an initiator for several other members of the caspase family and can lead to downstream mitochondrial damage. The naturally occurring human caspase 8 gene has nucleotide sequences as shown in Genbank Accession numbers: NM_001080124, NM_001080125, NM_001228, NM_033355, NM_033356 and the naturally occurring human caspase 8 protein has aminoacid sequences as shown in Genbank Accession numbers: NP_001073593, NP_001073594, NP_001219, NP_203519, NP_203520. The murine nucleotide and amino acid sequences have also been described (Genbank Accession numbers NM_001080126, NM_001277926, NM_009812 and NP_001073595, NP_001264855, NP_033942).

As used herein, the term “caspase 8 inhibitor” refers to a natural or synthetic compound that has a biological effect to inhibit the activity or the expression of caspase 8. More particularly, such compound is capable of inhibiting the protease activity of caspase 8. In the context of the invention, such compound is able to modify macrophage polarization in order to induce a pro-inflammatory environment. The method consists in the use of a caspase 8 inhibitor able to inhibit the polarization of anti-inflammatory M2-type macrophages and/or favors pro-inflammatory M1 -type macrophages, for inhibiting the anti-inflammatory signal provided by M2-type macrophages and favouring the pro-inflammatory signal provided by M1-type macrophages.

In a particular embodiment, the caspase 8 inhibitor is a peptide, peptidomimetic, small organic molecule, antibody, aptamers, siRNA or antisense oligonucleotide. The term “peptidomimetic” refers to a small protein-like chain designed to mimic a peptide. In a particular embodiment, the caspase 8 inhibitor is pan-Caspase inhibitor (Z-VAD-FMK), Caspase-1 Inhibitor I (Ac-YVAD-CHO), Caspase-8 Inhibitor II (Z-IETD-FMK), Caspase-3 Inhibitor II (Z-DEVD-FMK) and Caspase-9 Inhibitor (Z-LEHD-FMK).

In a particular embodiment, the caspase 8 inhibitor is an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity.

In a particular embodiment, the caspase 8 inhibitor is a small organic molecule. The term “small organic molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

In a particular embodiment, the caspase 8 inhibitor is a small molecule which is an selective inhibitor of caspase 8 selected among the following compounds: Emricasan, Nivocasan, Q-VD-OPh (1135695-98-5), PKR Inhibitor (CAS number: 608512-97-6), Q-VD-OPH (CAS 1135695-98-5), Gly-Phe β-naphthylamide (CAS number: 21438-66-4), BI-9B12 (CAS 848782-29-6).

In a particular embodiment, the caspase 8 inhibitor is Emricasan and its derivatives. As used herein, the term “Emricasan” also known as IDN-6556, 254750-02-2, PF-03491390, UNII-P0GMS9N47Q (S)-3 -((S)-2-(2-(2-TERT-BUTYLPHENYLAMINO)-2-OXOACETAMIDO)PROPANAMIDO)-4-OXO-5-(2,3,5,6-TETRAFLUOROPHENOXY)PENTANOIC ACID, PF 03491390, P0GMS9N47Q, (S)-3-((S)-2-(2-((2-(tert-Butyl)phenyl)amino)-2-oxoacetamido)propanamido)-4-oxo-5-(2,3,5,6-tetrafluorophenoxy)pentanoic acid refers to the first caspase inhibitor tested in human which has received orphan drug status by FDA. It is developed by Pfizer and made in such a way that it protects liver cells from excessive apoptosis. This molecule has the following formula, structure and the CAS number254750-02-2 in the art: C₂₆H₂₇F₄N₃O₇:

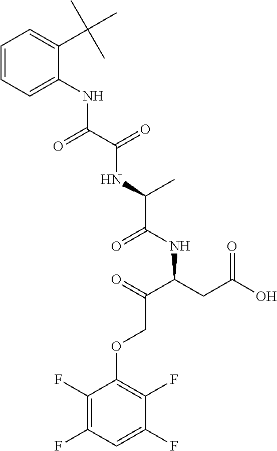 [SMILES: C[C@H](NC(=O)C(=O)Nc1ccccc1C(C)(C)C)C(=O)N[C@@H](CC(=O)O)C(=O)COc1c(F)c(F)cc(F)c1F]

In another embodiment, the caspase 8 inhibitor is Nivocasan and its derivatives. As used herein, the term “Nivocasan” also known as GS 9450 developed by Gilead Sciences, Inc (Ratziu V et al.2012; Arends JE et al.2011). Nivocasan has the following formula, structure and the CAS number 908253-63-4 in the art: C₂₁H₂₂FN₃O₅:

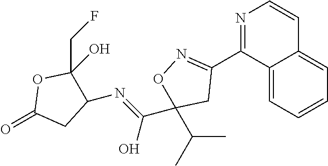 [SMILES: CC(C)C1(/C(O)=N/C2CC(=O)OC2(O)CF)CC(c2nccc3ccccc23)=NO1]

In some embodiments, the caspase 8 inhibitor is an antibody.

As used herein, the term “antibody” is used in the broadest sense and specifically covers monoclonal antibodies, polyclonal antibodies, multi specific antibodies (e.g. bispecific antibodies) formed from at least two intact antibodies, and antibody fragments so long as they exhibit the desired biological activity. The term includes antibody fragments that comprise an antigen binding domain such as Fab′, Fab, F(ab′)2, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art (see Kabat et al., 1991, specifically incorporated herein by reference). Diabodies, in particular, are further described in EP 404, 097 and WO 93/1 1 161; whereas linear antibodies are further described in Zapata et al. (1995). Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art. For example, each of Beckman et al., 2006; Holliger & Hudson, 2005; Le Gall et al., 2004; Reff & Heard, 2001; Reiter et al., 1996; and Young et al., 1995 further describe and enable the production of effective antibody fragments. In some embodiments, the antibody is a “chimeric” antibody as described in U.S. Pat. No. 4,816,567. In some embodiments, the antibody is a humanized antibody, such as described U.S. Pat. Nos. 6,982,321 and 7,087,409. In some embodiments, the antibody is a human antibody. A “human antibody” such as described in U.S. Pat. Nos. 6,075,181 and 6,150,584. In some embodiments, the antibody is a single domain antibody such as described in EP 0 368 684, WO 06/030220 and WO 06/003388. In a particular embodiment, the inhibitor is a monoclonal antibody. Monoclonal antibodies can be prepared and isolated using any technique that provides for the production of antibody molecules by continuous cell lines in culture. Techniques for production and isolation include but are not limited to the hybridoma technique, the human B-cell hybridoma technique and the EBV-hybridoma technique.

In a particular, the caspase 8 inhibitor is an intrabody having specificity for caspase 8. As used herein, the term “intrabody” generally refer to an intracellular antibody or antibody fragment. Antibodies, in particular single chain variable antibody fragments (scFv), can be modified for intracellular localization. Such modification may entail for example, the fusion to a stable intracellular protein, such as, e.g., maltose binding protein, or the addition of intracellular trafficking/localization peptide sequences, such as, e.g., the endoplasmic reticulum retention. In some embodiments, the intrabody is a single domain antibody. In some embodiments, the antibody according to the invention is a single domain antibody. The term “single domain antibody” (sdAb) or “VHH” refers to the single heavy chain variable domain of antibodies of the type that can be found in Camelid mammals which are naturally devoid of light chains. Such VHH are also called “nanobody®”. According to the invention, sdAb can particularly be llama sdAb.

In some embodiments, the inhibitor of caspase 8 expression is a short hairpin RNA (shRNA), a small interfering RNA (siRNA) or an antisense oligonucleotide which inhibits the expression of caspase 8. In a particular embodiment, the inhibitor of JMY expression is siRNA. A short hairpin RNA (shRNA) is a sequence of RNA that makes a tight hairpin turn that can be used to silence gene expression via RNA interference. shRNA is generally expressed using a vector introduced into cells, wherein the vector utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves mRNAs that match the siRNA to which it is bound. Small interfering RNA (siRNA), sometimes known as short interfering RNA or silencing RNA, are a class of 20-25 nucleotide-long double-stranded RNA molecules that play a variety of roles in biology. Most notably, siRNA is involved in the RNA interference (RNAi) pathway whereby the siRNA interferes with the expression of a specific gene. Anti-sense oligonucleotides include anti-sense RNA molecules and anti-sense DNA molecules, would act to directly block the translation of the targeted mRNA by binding thereto and thus preventing protein translation or increasing mRNA degradation, thus decreasing the level of the targeted protein, and thus activity, in a cell. For example, antisense oligonucleotides of at least about 15 bases and complementary to unique regions of the mRNA transcript sequence can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Antisense oligonucleotides, siRNAs, shRNAs of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and typically mast cells. Typically, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as Moloney murine leukaemia virus, Harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

In some embodiments, the inhibitor of caspase 8 expression is an endonuclease. In the last few years, staggering advances in sequencing technologies have provided an unprecedentedly detailed overview of the multiple genetic aberrations in cancer. By considerably expanding the list of new potential oncogenes and tumor suppressor genes, these new data strongly emphasize the need of fast and reliable strategies to characterize the normal and pathological function of these genes and assess their role, in particular as driving factors during oncogenesis. As an alternative to more conventional approaches, such as cDNA overexpression or downregulation by RNA interference, the new technologies provide the means to recreate the actual mutations observed in cancer through direct manipulation of the genome. Indeed, natural and engineered nuclease enzymes have attracted considerable attention in the recent years. The mechanism behind endonuclease-based genome inactivating generally requires a first step of DNA single or double strand break, which can then trigger two distinct cellular mechanisms for DNA repair, which can be exploited for DNA inactivating: the error prone nonhomologous end-joining (NHEJ) and the high-fidelity homology-directed repair (HDR).

In a particular embodiment, the endonuclease is CRISPR-cas. As used herein, the term “CRISPR-cas” has its general meaning in the art and refers to clustered regularly interspaced short palindromic repeats associated which are the segments of prokaryotic DNA containing short repetitions of base sequences.

In some embodiment, the endonuclease is CRISPR-cas9 which is from Streptococcus pyogenes. The CRISPR/Cas9 system has been described in U.S. Pat. No. 8,697,359 B1 and US 2014/0068797. Originally an adaptive immune system in prokaryotes (Barrangou and Marraffini, 2014), CRISPR has been recently engineered into a new powerful tool for genome editing. It has already been successfully used to target important genes in many cell lines and organisms, including human (Mali et al., 2013, Science, Vol. 339 : 823-826), bacteria (Fabre et al., 2014, PLoS Negl. Trop. Dis., Vol. 8:e2671.), zebrafish (Hwang et al., 2013, PLoS One, Vol. 8:e68708.), C. elegans (Hai et al., 2014 Cell Res. doi: 10.1038/cr.2014.11.), bacteria (Fabre et al., 2014, PLoS Negl. Trop. Dis., Vol. 8:e2671.), plants (Mali et al., 2013, Science, Vol. 339 : 823-826), Xenopus tropicalis (Guo et al., 2014, Development, Vol. 141 : 707-714.), yeast (DiCarlo et al., 2013, Nucleic Acids Res., Vol. 41 : 4336-4343.), Drosophila (Gratz et al., 2014 Genetics, doi:10.1534/genetics.113.160713), monkeys (Niu et al., 2014, Cell, Vol. 156 : 836-843.), rabbits (Yang et al., 2014, J. Mol. Cell Biol., Vol. 6: 97-99.), pigs (Hai et al., 2014, Cell Res. doi: 10.1038/cr.2014.11.), rats (Ma et al., 2014, Cell Res., Vol. 24: 122-125.) and mice (Mashiko et al., 2014, Dev. Growth Differ. Vol. 56 : 122-129.). Several groups have now taken advantage of this method to introduce single point mutations (deletions or insertions) in a particular target gene, via a single gRNA. Using a pair of gRNA-directed Cas9 nucleases instead, it is also possible to induce large deletions or genomic rearrangements, such as inversions or translocations. A recent exciting development is the use of the dCas9 version of the CRISPR/Cas9 system to target protein domains for transcriptional regulation, epigenetic modification, and microscopic visualization of specific genome loci.

In some embodiment, the endonuclease is CRISPR-Cpfl which is the more recently characterized CRISPR from Provotella and Francisella 1 (Cpf1) in Zetsche et al. (“Cpf1 is a Single RNA-guided Endonuclease of a Class 2 CRISPR-Cas System (2015); Cell; 163, 1-13).

### Method for Treating Macrophage Related Disease

Inventors have demonstrated Emricasan alleviates the IL4-mediated M2-like polarization of human macrophages. Moreover, Emricasan also hampers bleomycin-induced pulmonary fibrosis in mice, a disease associated with an infiltration of M2-macrophages. Finally, caspase-8 deficient mice were found to be resistant to bleomycin-induced pulmonary fibrosis. As a whole, their findings indicate that the beneficial effect of Emricasan relies on its ability to inhibit caspase-8, and its capacity to prevent monocyte differentiation and M2 polarization of macrophages.

Accordingly, in a second aspect, the invention relates to a caspase 8 inhibitor according to the invention for use as a drug.

In a particular embodiment, the caspase 8 inhibitor for use according to the invention in the treatment of macrophage related disease.

As used herein, the term “macrophage related disease” refers to diseases related to an undesirable M2 activation. In a particular embodiment, the caspase 8 inhibitor for use according to the invention wherein the macrophage related disease is selected from the group consisting of but not limited to: cancer, more particularly solid cancer, fibrotic diseases such as for example idiopathic pulmonary fibrosis (IPF), hepatic fibrosis or systemic sclerosis (Wynn and Barron, 2010, Semin. Liver Dis., 30, 245), allergy and asthma, atherosclerosis and Alzheimer's disease.

In a particular embodiment, the caspase 8 inhibitor for use according to the invention wherein the macrophage related disease is cancer.

As used herein, the term “cancer” refers to a malignant growth or tumor resulting from an uncontrolled division of cells. The term “cancer” includes primary tumors and metastatic tumors.

In a particular embodiment, the cancer is a solid cancer. In a particular embodiment, the solid cancer is selected from the group consisting of but not limited to: adrenal cortical cancer, anal cancer, bile duct cancer (e.g. peripheral cancer, distal bile duct cancer, intrahepatic bile duct cancer), bladder cancer, bone cancer (e.g. osteoblastoma, osteochondroma, hemangioma, chondromyxoid fibroma, osteosarcoma, chondrosarcoma, fibrosarcoma, malignant fibrous histiocytoma, giant cell tumor of the bone, chordoma, multiple myeloma), brain and central nervous system cancer (e.g. meningioma, astrocytoma, oligodendrogliomas, ependymoma, gliomas, medulloblastoma, ganglioglioma, Schwannoma, germinoma, craniopharyngioma), breast cancer (e.g. ductal carcinoma in situ, infiltrating ductal carcinoma, infiltrating lobular carcinoma, lobular carcinoma in situ, gynecomastia), cervical cancer, colorectal cancer, endometrial cancer (e.g. endometrial adenocarcinoma, adenoacanthoma, papillary serous adenocarcinoma, clear cell), esophagus cancer, gallbladder cancer (mucinous adenocarcinoma, small cell carcinoma), gastrointestinal carcinoid tumors (e.g. choriocarcinoma, chorioadenoma destruens), Kaposi's sarcoma, kidney cancer (e.g. renal cell cancer), laryngeal and hypopharyngeal cancer, liver cancer (e.g. hemangioma, hepatic adenoma, focal nodular hyperplasia, hepatocellular carcinoma), lung cancer (e.g. small cell lung cancer, non-small cell lung cancer), mesothelioma, plasmacytoma, nasal cavity and paranasal sinus cancer (e.g. esthesioneuroblastoma, midline granuloma), nasopharyngeal cancer, neuroblastoma, oral cavity and oropharyngeal cancer, ovarian cancer, pancreatic cancer, penile cancer, pituitary cancer, prostate cancer, retinoblastoma, rhabdomyosarcoma (e.g. embryonal rhabdomyosarcoma, alveolar rhabdomyosarcoma, pleomorphic rhabdomyosarcoma), salivary gland cancer, skin cancer (e.g. melanoma, nonmelanoma skin cancer), stomach cancer, testicular cancer (e.g. seminoma, nonseminoma germ cell cancer), thymus cancer, thyroid cancer (e.g. follicular carcinoma, anaplastic carcinoma, poorly differentiated carcinoma, medullary thyroid carcinoma), vaginal cancer, vulvar cancer, and uterine cancer (e.g. uterine leiomyosarcoma).

In a particular embodiment, the solid cancer is melanoma.

In another embodiment, the solid cancer is liver cancer. More particularly, in a particular embodiment the liver cancer is hepatocellular carcinoma (HCC).

In a further embodiment, the caspase 8 inhibitor for use according to the invention wherein the macrophage related disease is fibrosis.

As used herein, the term “fibrosis” refers to the common scarring reaction associated with chronic injury that results from prolonged parenchymal cell injury and/or inflammation that may be induced by a wide variety of agents, e.g., drugs, toxins, radiation, any process disturbing tissue or cellular homeostasis, toxic injury, altered blood flow, infections (viral, bacterial, spirochetal, and parasitic), storage disorders, and disorders resulting in the accumulation of toxic metabolites. Fibrosis is most common in the heart, lung, peritoneum, and kidney.

In a particular embodiment, the fibrosis affects at least one organ selected from the group consisting of skin, heart, liver, lung, or kidney. Examples of fibrosis include, without limitation, dermal scar formation, keloids, liver fibrosis, lung fibrosis, kidney fibrosis, glomerulosclerosis, pulmonary fibrosis (e.g. idiopathic pulmonary fibrosis), liver fibrosis (e.g. following liver transplantation, liver fibrosis following chronic hepatitis C virus infection), renal fibrosis, intestinal fibrosis, interstitial fibrosis, cystic fibrosis of the pancreas and lungs, injection fibrosis, endomyocardial fibrosis, mediastinal fibrosis, myelofibrosis, retroperitoneal fibrosis, progressive massive fibrosis, nephrogenic systemic fibrosis . . . In some embodiments, the fibrosis is caused by surgical implantation of an artificial organ. In a particular embodiment, the fibrosis is lung fibrosis.

In a particular embodiment, the caspase 8 inhibitor for use according to the invention is Emricasan as described above.

In a particular embodiment, the invention relates to a method for treating macrophage related disease in a subject in need thereof comprising a step of administering the subject with a therapeutically effective amount of a caspase 8 inhibitor.

As used herein, the terms “treating” or “treatment” refer to both prophylactic or preventive treatment as well as curative or disease modifying treatment, including treatment of subject at risk of contracting the disease or suspected to have contracted the disease as well as subject who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “subject” refers to any mammals, such as a rodent, a feline, a canine, and a primate. Particularly, in the present invention, the subject is a human afflicted with or susceptible to be afflicted with macrophages related disease. In another embodiment, the subject is a human afflicted with or susceptible to be afflicted with a cancer. In another embodiment, the subject is a human afflicted with or susceptible to be afflicted with a solid cancer. In another embodiment, the subject is a human afflicted with or susceptible to be afflicted with melanoma. In another embodiment, the subject is a human afflicted with or susceptible to be afflicted with HCC. In another embodiment, the subject is a human afflicted with or susceptible to be afflicted with a fibrosis. In another embodiment, the subject is a human afflicted with or susceptible to be afflicted with lung fibrosis.

The present invention also relates to a method for treating macrophages related disease in a subject in need thereof comprising a step of administering the subject with a therapeutically effective amount of a caspase 8 inhibitor. In a particular embodiment, the method according to the invention, wherein the caspase 8 inhibitor and a classical treatment, as combined preparation for use simultaneously, separately or sequentially in the treatment of macrophages related disease.

In another embodiment, the invention relates to a combined preparation comprising the caspase 8 inhibitor for use according to the invention and a classical treatment. More particularly, the invention relates to a i) caspase 8 inhibitor and a ii) classical treatment for simultaneous, separate or sequential use in the treatment of macrophages related disease, as a combined preparation.

In a particular embodiment, the invention relates to an i) caspase 8 inhibitor and ii) a classical treatment for simultaneous, separate or sequential use in the treatment of a solid cancer.

In a particular embodiment, the invention relates to an i) caspase 8 inhibitor and ii) a classical treatment for simultaneous, separate or sequential use in the treatment of melanoma.

In a particular embodiment, the invention relates to an i) caspase 8 inhibitor and ii) a classical treatment for simultaneous, separate or sequential use in the treatment of HCC.

In a particular embodiment, the invention relates to an i) caspase 8 inhibitor and ii) a classical treatment for simultaneous, separate or sequential use in the treatment of fibrosis.

In a particular embodiment, the invention relates to an i) caspase 8 inhibitor and ii) a classical treatment for simultaneous, separate or sequential use in the treatment of lung fibrosis.

As used herein, the term “classical treatment” refers to any compound, natural or synthetic, and immunotherapy, chemotherapy and radiotherapy used for the treatment of a cancer.

In a particular embodiment, the classical treatment refers to a treatment with a chemotherapeutic agent.

Typically, the invention relates to an i) caspase 8 inhibitor and ii) a chemotherapeutic agent for simultaneous, separate or sequential use in the treatment of a solid cancer such as melanoma or HCC.

As used herein, the term “chemotherapeutic agent” refers to chemical compounds that are effective in inhibiting tumor growth. Examples of chemotherapeutic agents include alkylating agents such as thiotepa and cyclophosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaorarnide and trimethylolomelamine; acetogenins (especially bullatacin and bullatacinone); a carnptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estrarnustine, ifosfamide, mechlorethamine, mechlorethamine oxide hydrochloride, melphalan, novembichin, phenesterine, prednimus tine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin (11 and calicheamicin 211, see, e.g., Agnew Chem Intl. Ed. Engl. 33: 183-186 (1994); dynemicin, including dynemicin A; an esperamicin; as well as neocarzinostatin chromophore and related chromoprotein enediyne antiobiotic chromomophores), aclacinomysins, actinomycin, authramycin, azaserine, bleomycins, cactinomycin, carabicin, canninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idanrbicin, marcellomycin, mitomycins, mycophenolic acid, nogalarnycin, olivomycins, peplomycin, potfiromycin, puromycin, quelamycin, rodorubicin, streptomgrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophospharnide glycoside; aminolevulinic acid; amsacrine; bestrabucil; bisantrene; edatraxate; defo famine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pento statin; phenamet; pirarubicin; podophyllinic acid; 2-ethylhydrazide; procarbazine; PSK®; razoxane; rhizoxin; sizofiran; spirogennanium; tenuazonic acid; triaziquone; 2,2′,2″-trichlorotriethylarnine; trichothecenes (especially T-2 toxin, verracurin A, roridinA and anguidine); urethan; vindesine; dacarbazine; mannomustine; mitobromtol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.J.) and doxetaxel (TAXOTERE®, Rhone-Poulenc Rorer, Antony, France); chlorambucil; gemcitabine; 6-thioguanine; mercaptopurine; methotrexate; platinum analogs such as cisp latin and carbop latin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-1 1; topoisomerase inhibitor RFS 2000; difluoromethylornithine (DMFO); retinoic acid; capecitabine; and pharmaceutically acceptable salts, acids or derivatives of any of the above. Also included in this definition are antihormonal agents that act to regulate or inhibit honnone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

In a particular embodiment, the classical treatment refers to a targeted therapy (TT).

Typically, the invention relates to an i) caspase 8 inhibitor and ii) a targeted therapy for simultaneous, separate or sequential use in the treatment of a solid cancer such as melanoma or HCC.

As used herein, the term “targeted therapy” refers to targeting the cancer's specific genes, proteins, or the tissue environment that contributes to cancer growth and survival. Example of targeted therapy: targeting human epidermal growth factor receptor 2 (HER2) for breast cancer; targeting epidermal growth factor receptor (EGFR), or vascular endothelial growth factor (VEGF) for colorectal cancer or lung cancer; targeting BRAF for melanoma.

In a particular embodiment, the classical treatment refers to a treatment with an immunotherapeutic agent.

Typically, the invention relates to an i) caspase 8 inhibitor and ii) an immunotherapeutic agent for simultaneous, separate or sequential use in the treatment of a solid cancer such as melanoma or HCC.

The term “immunotherapeutic agent” as used herein, refers to a compound, composition or treatment that indirectly or directly enhances, stimulates or increases the body's immune response against cancer cells and/or that decreases the side effects of other anticancer therapies. Immunotherapy is thus a therapy that directly or indirectly stimulates or enhances the immune system's responses to cancer cells and/or lessens the side effects that may have been caused by other anti-cancer agents. Immunotherapy is also referred to in the art as immunologic therapy, biological therapy biological response modifier therapy and biotherapy. Examples of common immunotherapeutic agents known in the art include, but are not limited to, immune checkpoint inhibitor, cytokines, cancer vaccines, monoclonal antibodies and non-cytokine adjuvants. Alternatively, the immunotherapeutic treatment may consist of administering the subject with an amount of immune cells (T cells, NK, cells, dendritic cells, B cells . . . ). Immunotherapeutic agents can be non-specific, i.e. boost the immune system generally so that the human body becomes more effective in fighting the growth and/or spread of cancer cells, or they can be specific, i.e. targeted to the cancer cells themselves immunotherapy regimens may combine the use of non-specific and specific immunotherapeutic agents. Non-specific immunotherapeutic agents are substances that stimulate or indirectly improve the immune system. Non-specific immunotherapeutic agents have been used alone as a main therapy for the treatment of cancer, as well as in addition to a main therapy, in which case the non-specific immunotherapeutic agent functions as an adjuvant to enhance the effectiveness of other therapies (e.g. cancer vaccines). Non-specific immunotherapeutic agents can also function in this latter context to reduce the side effects of other therapies, for example, bone marrow suppression induced by certain chemotherapeutic agents. Non-specific immunotherapeutic agents can act on key immune system cells and cause secondary responses, such as increased production of cytokines and immunoglobulins. Alternatively, the agents can themselves comprise cytokines. Non-specific immunotherapeutic agents are generally classified as cytokines or non-cytokine adjuvants. A number of cytokines have found application in the treatment of cancer either as general non-specific immunotherapies designed to boost the immune system, or as adjuvants provided with other therapies. Suitable cytokines include, but are not limited to, interferons, interleukins and colony-stimulating factors. Interferons (IFNs) contemplated by the present invention include the common types of IFNs, IFN-alpha (IFN-α), and IFN-beta (IFN-β). IFNs can act directly on cancer cells, for example, by slowing their growth, promoting their development into cells with more normal behaviour and/or increasing their production of antigens thus making the cancer cells easier for the immune system to recognise and destroy. IFNs can also act indirectly on cancer cells, for example, by slowing down angiogenesis, boosting the immune system and/or stimulating natural killer (NK) cells, T cells and macrophages. Recombinant IFN-alpha is available commercially as Roferon (Roche Pharmaceuticals) and Intron A (Schering Corporation). Interleukins contemplated by the present invention include IL-2, IL-4, IL-11 and IL-12. Examples of commercially available recombinant interleukins include Proleukin® (IL-2; Chiron Corporation) and Neumega® (IL-12; Wyeth Pharmaceuticals). Zymogenetics, Inc. (Seattle, Wash.) is currently testing a recombinant form of IL-21, which is also contemplated for use in the combinations of the present invention. Colony-stimulating factors (CSFs) contemplated by the present invention include sargramostim. Treatment with one or more growth factors can help to stimulate the generation of new blood cells in subjects undergoing traditional chemotherapy. Accordingly, treatment with CSFs can be helpful in decreasing the side effects associated with chemotherapy and can allow for higher doses of chemotherapeutic agents to be used. In addition to having specific or non-specific targets, immunotherapeutic agents can be active, i.e. stimulate the body's own immune response, or they can be passive, i.e. comprise immune system components that were generated external to the body. Passive specific immunotherapy typically involves the use of one or more monoclonal antibodies that are specific for a particular antigen found on the surface of a cancer cell or that are specific for a particular cell growth factor. Monoclonal antibodies may be used in the treatment of cancer in a number of ways, for example, to enhance a subject's immune response to a specific type of cancer, to interfere with the growth of cancer cells by targeting specific cell growth factors, such as those involved in angiogenesis, or by enhancing the delivery of other anticancer agents to cancer cells when linked or conjugated to agents such as chemotherapeutic agents, radioactive particles or toxins. Monoclonal antibodies currently used as cancer immunotherapeutic agents that are suitable for inclusion in the combinations of the present invention include, but are not limited to, rituximab (Rituxan®), trastuzumab (Herceptin®), ibritumomab tiuxetan (Zevalin®), tositumomab (Bexxar®), cetuximab (C-225, Erbitux®), bevacizumab (Avastin®), gemtuzumab ozogamicin (Mylotarg®), alemtuzumab (Campath®), and BL22. Other examples include anti-CTLA4 antibodies (e.g. Ipilimumab), anti-PD1 antibodies, anti-PDL1 antibodies, anti-PLD2 antibodies, anti-TIMP3 antibodies, anti-LAG3 antibodies, anti-B7H3 antibodies, anti-B7H4 antibodies or anti-B7H6 antibodies. In some embodiments, antibodies include B cell depleting antibodies. Typical B cell depleting antibodies include but are not limited to anti-CD20 monoclonal antibodies [e.g. Rituximab (Roche), Ibritumomab tiuxetan (Bayer Schering), Tositumomab (GlaxoSmithKline), AME-133v (Applied Molecular Evolution), Ocrelizumab (Roche), Ofatumumab (HuMax-CD20, Gemnab), TRU-015 (Trubion) and IMMU-106 (Immunomedics)], an anti-CD22 antibody [e.g. Epratuzumab, Leonard et al., Clinical Cancer Research (Z004) 10: 53Z7-5334], anti-CD79a antibodies, anti-CD27 antibodies, or anti-CD19 antibodies (e.g. U.S. Pat. No. 7,109,304), anti-BAFF-R antibodies (e.g. Belimumab, GlaxoSmithKline), anti-APRIL antibodies (e.g. anti-human APRIL antibody, ProSci inc.), and anti-IL-6 antibodies [e.g. previously described by De Benedetti et al., J Immunol (2001) 166: 4334-4340 and by Suzuki et al., Europ J of Immunol (1992) 22 (8) 1989-1993, fully incorporated herein by reference]. The immunotherapeutic treatment may consist of allografting, in particular, allograft with hematopoietic stem cell HSC. The immunotherapeutic treatment may also consist in an adoptive immunotherapy as described by Nicholas P. Restifo, Mark E. Dudley and Steven A. Rosenberg “Adoptive immunotherapy for cancer: harnessing the T cell response, Nature Reviews Immunology, Volume 12, April 2012). In adoptive immunotherapy, the subject's circulating lymphocytes, NK cells, are isolated amplified in vitro and readministered to the subject. The activated lymphocytes or NK cells are most particularly be the subject's own cells that were earlier isolated from a blood or tumor sample and activated (or “expanded”) in vitro.

In a particular embodiment, the classical treatment refers to a treatment with an immune checkpoint inhibitor.

Typically, the invention relates to an i) caspase 8 inhibitor and ii) an immune checkpoint inhibitor for simultaneous, separate or sequential use in the treatment of a solid cancer such as melanoma or HCC.

As used herein, the term “immune checkpoint inhibitor” refers to molecules that totally or partially reduce, inhibit, interfere with or modulate one or more immune checkpoint proteins.

As used herein, the term “immune checkpoint protein” has its general meaning in the art and refers to a molecule that is expressed by T cells in that either turn up a signal (stimulatory checkpoint molecules) or turn down a signal (inhibitory checkpoint molecules). Immune checkpoint molecules are recognized in the art to constitute immune checkpoint pathways similar to the CTLA-4 and PD-1 dependent pathways (see e.g. Pardoll, 2012. Nature Rev Cancer 12:252-264; Mellman et al. , 2011. Nature 480:480-489). Examples of stimulatory checkpoint include CD27 CD28 CD40, CD122, CD137, OX40, GITR, and ICOS. Examples of inhibitory checkpoint molecules include A2AR, B7-H3, B7-H4, BTLA, CTLA-4, CD277, IDO, KIR, PD-1, LAG-3, TIM-3 and VISTA. The Adenosine A2A receptor (A2AR) is regarded as an important checkpoint in cancer therapy because adenosine in the immune microenvironment, leading to the activation of the A2a receptor, is negative immune feedback loop and the tumor microenvironment has relatively high concentrations of adenosine. B7-H3, also called CD276, was originally understood to be a co-stimulatory molecule but is now regarded as co-inhibitory. B7-H4, also called VTCN1, is expressed by tumor cells and tumor-associated macrophages and plays a role in tumour escape. B and T Lymphocyte Attenuator (BTLA) and also called CD272, has HVEM (Herpesvirus Entry Mediator) as its ligand. Surface expression of BTLA is gradually downregulated during differentiation of human CD8+ T cells from the naive to effector cell phenotype, however tumor-specific human CD8+ T cells express high levels of BTLA. CTLA-4, Cytotoxic T-Lymphocyte-Associated protein 4 and also called CD152. Expression of CTLA-4 on Treg cells serves to control T cell proliferation. IDO, Indoleamine 2,3-dioxygenase, is a tryptophan catabolic enzyme. A related immune-inhibitory enzymes. Another important molecule is TDO, tryptophan 2,3-dioxygenase. IDO is known to suppress T and NK cells, generate and activate Tregs and myeloid-derived suppressor cells, and promote tumour angiogenesis. KIR, Killer-cell Immunoglobulin-like Receptor, is a receptor for MHC Class I molecules on Natural Killer cells. LAG3, Lymphocyte Activation Gene-3, works to suppress an immune response by action to Tregs as well as direct effects on CD8+ T cells. PD-1, Programmed Death 1 (PD-1) receptor, has two ligands, PD-L1 and PD-L2. This checkpoint is the target of Merck & Co.'s melanoma drug Keytruda, which gained FDA approval in September 2014. An advantage of targeting PD-1 is that it can restore immune function in the tumor microenvironment. TIM-3, short for T-cell Immunoglobulin domain and Mucin domain 3, expresses on activated human CD4+ T cells and regulates Th1 and Th17 cytokines. TIM-3 acts as a negative regulator of Th1/Tc1 function by triggering cell death upon interaction with its ligand, galectin-9. VISTA, Short for V-domain Ig suppressor of T cell activation, VISTA is primarily expressed on hematopoietic cells so that consistent expression of VISTA on leukocytes within tumors may allow VISTA blockade to be effective across a broad range of solid tumors. Tumor cells often take advantage of these checkpoints to escape detection by the immune system. Thus, inhibiting a checkpoint protein on the immune system may enhance the anti-tumor T-cell response.

In some embodiments, an immune checkpoint inhibitor refers to any compound inhibiting the function of an immune checkpoint protein. Inhibition includes reduction of function and full blockade. In some embodiments, the immune checkpoint inhibitor could be an antibody, synthetic or native sequence peptides, small molecules or aptamers which bind to the immune checkpoint proteins and their ligands.

In a particular embodiment, the immune checkpoint inhibitor is an antibody.

Typically, antibodies are directed against A2AR, B7-H3, B7-H4, BTLA, CTLA-4, CD277, IDO, KIR, PD-1, LAG-3, TIM-3 or VISTA.

In a particular embodiment, the immune checkpoint inhibitor is an anti-PD-1 antibody such as described in WO2011082400, WO2006121168, WO2015035606, WO2004056875, WO2010036959, WO2009114335, WO2010089411, WO2008156712, WO2011110621, WO2014055648 and WO2014194302. Examples of anti-PD-1 antibodies which are commercialized: Nivolumab (Opdivo®, BMS), Pembrolizumab (also called Lambrolizumab, KEYTRUDA® or MK-3475, MERCK).

In some embodiments, the immune checkpoint inhibitor is an anti-PD-L1 antibody such as described in WO2013079174, WO2010077634, WO2004004771, WO2014195852, WO2010036959, WO2011066389, WO2007005874, WO2015048520, U.S. Pat. No. 8,617,546 and WO2014055897. Examples of anti-PD-L1 antibodies which are on clinical trial: Atezolizumab (MPDL3280A, Genentech/Roche), Durvalumab (AZD9291, AstraZeneca), Avelumab (also known as MSB0010718C, Merck) and BMS-936559 (BMS).

In some embodiments, the immune checkpoint inhibitor is an anti-PD-L2 antibody such as described in U.S. Pat. Nos. 7,709,214, 7,432,059 and 8,552,154.

In the context of the invention, the immune checkpoint inhibitor inhibits Tim-3 or its ligand.

In a particular embodiment, the immune checkpoint inhibitor is an anti-Tim-3 antibody such as described in WO03063792, WO2011155607, WO2015117002, WO2010117057 and WO2013006490.

In some embodiments, the immune checkpoint inhibitor is a small organic molecule.

The term “small organic molecule” as used herein, refers to a molecule of a size comparable to those organic molecules generally used in pharmaceuticals. The term excludes biological macro molecules (e. g. proteins, nucleic acids, etc.). Typically, small organic molecules range in size up to about 5000 Da, more preferably up to 2000 Da, and most preferably up to about 1000 Da.

Typically, the small organic molecules interfere with transduction pathway of A2AR, B7-H3, B7-H4, BTLA, CTLA-4, CD277, IDO, KIR, PD-1, LAG-3, TIM-3 or VISTA.

In a particular embodiment, small organic molecules interfere with transduction pathway of PD-1 and Tim-3. For example, they can interfere with molecules, receptors or enzymes involved in PD-1 and Tim-3 pathway.

In a particular embodiment, the small organic molecules interfere with Indoleamine-pyrrole 2,3-dioxygenase (IDO) inhibitor. IDO is involved in the tryptophan catabolism (Liu et al 2010, Vacchelli et al 2014, Zhai et al 2015). Examples of IDO inhibitors are described in WO 2014150677. Examples of IDO inhibitors include without limitation 1-methyl-tryptophan (IMT), β-(3-benzofuranyl)-alanine, β-(3-benzo(b)thienyl)-alanine), 6-nitro-tryptophan, 6-fluoro-tryptophan, 4-methyl-tryptophan, 5 -methyl tryptophan, 6-methyl-tryptophan, 5-methoxy-tryptophan, 5 -hydroxy-tryptophan, indole 3-carbinol, 3,3′-diindolylmethane, epigallocatechin gallate, 5-Br-4-Cl-indoxyl 1,3-diacetate, 9-vinylcarbazole, acemetacin, 5-bromo-tryptophan, 5-bromoindoxyl diacetate, 3-Amino-naphtoic acid, pyrrolidine dithiocarbamate, 4-phenylimidazole a brassinin derivative, a thiohydantoin derivative, a β-carboline derivative or a brassilexin derivative. In a particular embodiment, the IDO inhibitor is selected from 1-methyl-tryptophan, β-(3-benzofuranyl)-alanine, 6-nitro-L-tryptophan, 3-Amino-naphtoic acid and β-[3-benzo(b)thienyl]-alanine or a derivative or prodrug thereof.

In a particular embodiment, the inhibitor of IDO is Epacadostat, (INCB24360, INCB024360) has the following chemical formula in the art and refers to -N-(3-bromo-4-fluorophenyl)-N′-hydroxy-4-{[2-(sulfamoylamino)-éthyl]amino}-1,2,5-oxadiazole-3 carboximidamide:

In a particular embodiment, the inhibitor is BGB324, also called R428, such as described in WO2009054864, refers to 1H-1,2,4-Triazole-3,5-diamine, 1-(6,7-dihydro-5H-benzo[6,7]cyclohepta[1,2-c]pyridazin-3-yl)-N3-[(7S)-6,7,8,9-tetrahydro-7-(1-pyrrolidinyl)-5H-benzocyclohepten-2-yl]- and has the following formula in the art:

In a particular embodiment, the inhibitor is CA-170 (or AUPM-170): an oral, small molecule immune checkpoint antagonist targeting programmed death ligand-1 (PD-L1) and V-domain Ig suppressor of T cell activation (VISTA) (Liu et al 2015). Preclinical data of CA-170 are presented by Curis Collaborator and Aurigene on November at ACR-NCI-EORTC International Conference on Molecular Targets and Cancer Therapeutics.

In some embodiments, the immune checkpoint inhibitor is an aptamer.

Typically, the aptamers are directed against A2AR, B7-H3, B7-H4, BTLA, CTLA-4, CD277, IDO, KIR, PD-1, LAG-3, TIM-3 or VISTA.

In a particular embodiment, aptamers are DNA aptamers such as described in Prodeus et al 2015. A major disadvantage of aptamers as therapeutic entities is their poor pharmacokinetic profiles, as these short DNA strands are rapidly removed from circulation due to renal filtration. Thus, aptamers according to the invention are conjugated to with high molecular weight polymers such as polyethylene glycol (PEG). In a particular embodiment, the aptamer is an anti-PD-1 aptamer. Particularly, the anti-PD-1 aptamer is MP7 pegylated as described in Prodeus et al 2015.

As used herein the terms “administering” or “administration” refer to the act of injecting or otherwise physically delivering a substance as it exists outside the body (e.g., an inhibitor of caspase 8 alone or in a combination with a classical treatment) into the subject, such as by, intravenous, intramuscular, enteral, subcutaneous, parenteral, systemic, local, spinal, nasal, topical or epidermal administration (e.g., by injection or infusion). When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

A “therapeutically effective amount” is intended for a minimal amount of active agent which is necessary to impart therapeutic benefit to a subject. For example, a “therapeutically effective amount” to a subject is such an amount which induces, ameliorates or otherwise causes an improvement in the pathological symptoms, disease progression or physiological conditions associated with or resistance to succumbing to a disorder. It will be understood that the total daily usage of the compounds of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidential with the specific compound employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day. In a particular embodiment, Emricasan is administered orally between 5 and 50 mg twice per day. In a particular embodiment, Nivocasan is administered orally between 10 and 80 mg per day.

In a third aspect, the invention relates to a pharmaceutical for use in the treatment of macrophages related disease.

In a particular embodiment, the pharmaceutical composition according to the invention comprises a caspase 8 inhibitor.

In a particular embodiment, the invention relates to a pharmaceutical composition comprising a caspase 8 inhibitor and a classical treatment as described above.

In a particular embodiment, the pharmaceutical composition according to the invention wherein the caspase 8 inhibitor and a classical treatment, as combined preparation for use simultaneously, separately or sequentially in the treatment of macrophages related disease.

In another embodiment, the pharmaceutical composition according to the invention, wherein the caspase 8 inhibitor is Emricasan.

The caspase 8 inhibitor as described above may be combined with pharmaceutically acceptable excipients, and optionally sustained-release matrices, such as biodegradable polymers, to form pharmaceutical compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The polypeptide (or nucleic acid encoding thereof) can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

In certain embodiments, the pharmaceutical formulation can be suitable for parenteral administration. The terms “parenteral administration” and “administered parenterally,” as used herein, refers to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. In certain embodiments, the present invention provides a parenteral formulation comprising a caspase 8 inhibitor and a classical as a combined preparation. In certain embodiments, the present invention provides a parenteral formulation comprising a caspase 8 inhibitor and a classical treatment as a combined preparation. For example, and not by way of limitation, the present invention provides a parenteral formulation comprising Emricasan and a classical treatment as a combined preparation. In a particular embodiment, when the caspase 8 inhibitor is combined with a classical treatment, the combination is formulated for oral, cutaneous or topical use.

### Method of Screening a Caspase 8 Inhibitor

A further object of the present invention relates to a method of screening a drug suitable for the treatment of macrophage related disease comprising i) providing a test compound and ii) determining the ability of said test compound to inhibit the activity and/or expression of caspase 8.

Any biological assay well known in the art could be suitable for determining the ability of the test compound to inhibit the activity of caspase 8. In some embodiments, the assay first comprises determining the ability of the test compound to bind to caspase 8. In some embodiments, a population of cells is then contacted and activated so as to determine the ability of the test compound to inhibit the activity of caspase 8. In particular, the effect triggered by the test compound is determined relative to that of a population of immune cells incubated in parallel in the absence of the test compound or in the presence of a control agent either of which is analogous to a negative control condition. The term “control substance”, “control agent”, or “control compound” as used herein refers a molecule that is inert or has no activity relating to an ability to modulate a biological activity or expression. It is to be understood that test compounds capable of inhibiting the activity of caspase 8, as determined using in vitro methods described herein, are likely to exhibit similar modulatory capacity in applications in vivo. Typically, the test compound is selected from the group consisting of peptides, peptidomimetics, small organic molecules, aptamers or nucleic acids. For example the test compound according to the invention may be selected from a library of compounds previously synthesised, or a library of compounds for which the structure is determined in a database, or from a library of compounds that have been synthesised de novo. In some embodiments, the test compound may be selected form small organic molecules.

The invention will be further illustrated by the following figures and examples. However, these examples and figures should not be interpreted in any way as limiting the scope of the present invention.

### FIGURES

FIG. 1. Emricasan inhibits CSF-1-induced monocyte differentiation at the micromolar level. Human peripheral blood monocytes from healthy donors were exposed for 2 days to 100 ng/mL CSF-1 alone or in combination with indicated concentrations (μM) of Emricasan which was added 30 min before CSF-1 treatment. (A) Macrophagic differentiation of monocytes from 3 different healthy donors was examined by 3-color flow cytometric analysis. The results are expressed as percentage of CD71/CD163 or CD16/CD163 double positive cells and represent the mean±SD of 3 independent experiments performed in duplicate. (B) Cell death from 3 different healthy donors was examined by flow cytometry analysis. The results are expressed as percentage of AnnexinV/DAPI double positive cells and represent the mean±SD of 3 independent experiments performed in duplicate. n.s. denotes not statistically significant according to a paired student t test. *P<0.05, **P<0.01, ***P<0.001 according to a paired student t test (versus d2).

FIG. 2. Emricasan is a more effective inhibitor of CSF-1-induced monocyte differentiation compared to Q-VD-OPh. Human blood monocytes were exposed for 2 days to 100 ng/mL CSF-1 alone or in combination with indicated concentrations of Emricasan or Q-VD-OPh (qVD) which were added 30 min before CSF-1 treatment. Macrophagic differentiation of monocytes from 3 different healthy donors was examined by 3-color flow cytometric analysis. The results are expressed as percentage of CD71/CD163 or CD16/CD163 double positive cells and represent the mean±SD of 3 independent experiments performed in duplicate. n.s. denotes not statistically significant according to a paired student t test. *P<0.05, **P<0.01, ***P<0.001 according to a paired student t test (versus d2).

FIG. 3. Both Q-VD-Oph and Emricasan hamper in the same way apoptosis in untreated monocytes. Human blood monocytes were exposed for 1 day to indicated concentrations of Q-VD-OPh (qVD) or Emricasan. Measures of caspase-3 (DEVD-AMC) and caspase-8 (IETD-AMC) activities. The results are expressed as A.U./mg/min and represent the mean the mean±SD of 3 independent experiments performed in triplicate.

FIG. 4. Emricasan is an effective inhibitor of caspase-8 and caspase-3. The ability of Emricasan or Q-VD-OPh (qVD) to inhibit caspases activities were assessed using active recombinant proteins of caspase-8 and caspase-3. (A) Measures of caspase-8 activity (IETD-AMC). IETD-CHO treatment is used as positive control in the in vitro assay. The results are expressed as A.U./min and represent the mean of 3 independent experiments realized in duplicate (B) Measures of caspase-3 activity (DEVD-AMC). DEVD-CHO is used as positive control in the in vitro assay. The results are expressed as A.U./min and represent the mean of 3 independent experiments realized in duplicate.

FIG. 5. Emricasan is a potent inhibitor of CSF-1-induced caspases activation. Human blood monocytes were exposed for 2 days or 3 days to 100 ng/mL CSF-1 alone or in combination with indicated concentrations of Emricasan or Q-VD-OPh (qVD) which were added 30 min before CSF-1 treatment. Caspases activities from 3 different healthy donors was examined by flow cytometry analysis. The results are expressed as percentage of IETD or DEVD positive cells and represent the mean±SD of 3 independent experiments performed in duplicate. n.s. denotes not statistically significant according to a paired student t test. *P<0.05, **P<0.01, ***P<0.001 according to a paired student t test (versus d2). Asterisks indicate cleavage fragments. Each panel is representative of at least 3 independent experiments.

FIG. 6. Emricasan blocks the M2-polarization of CSF-1-derived macrophages. Human monocytes were differentiated during 7 days with 100 ng/mL CSF-1. Emricasan was added 60h before the end of CSF-1 treatment. (A) Functional assay of CSF-1-derived macrophages exposed for 7 days to 100 ng/mL CSF- with or without Emricasan. The results are expressed as MFI and represent the mean of 3 independent experiments performed in duplicate. **P<0.01 according to a paired student t test (B) Macrophage polarization was evaluated by 3-color flow cytometric analysis. The percentage indicates cells that express both CD206/CD200R or CD163/CD200R. The results represent the mean±SD of 3 independent experiments performed in duplicate. **P<0.01, ***P<0.001 according to a paired student t test. Human monocytes were differentiated during 5 days with 100 ng/mL CSF-1 and then polarized into M2-macrophages (IL-4) for 2 days. Emricasan was added 16 h before the IL-4 treatment. The results are expressed as percentage of CD200R/CD206 or CD200R/CD163 double positive cells and represent the mean±SD of 3 independent experiments performed in duplicate.

FIG. 7. Pharmacologic and genetic inhibition of caspase-8 prevents bleomycin-induced pulmonary fibrosis. (A) Quantification of Sirius Red labeling intensity. Results are expressed as fold change in Sirius Red staining in treated compared to control mice (bleomycin was compared to untreated, bleomycin+Emricsan to Emricasan alone). Each dot or square is an individual mouse. *P<0.05 according to Mann-Whitney test. (B) Quantification of airspace number/mm2 of parenchymal tissue. Results expressed as fold change in treated compared to control mice as in B. *P<0.05 according to Mann-Whitney test. (C) Quantification of Sirius Red labeling intensity. Results expressed as fold change in treated compared to control mice, C8 KO+bleomycin compared to C8 KO as in B. *P<0.05 according to Mann-Whitney test. (D) Quantification of airspace number/mm2 of parenchymal tissue. Results are expressed as fold change in treated compared to untreated wild-type mice, as in panel E. *P<0.05 according to Mann-Whitney test. (E) Cytokines were measured in broncho-alveolar lavage fluid collected from bleomycin-treated wild-type (wt) and LysM-Cre/Caspase-8 flox/flox (C8 KO) mice treated with bleomycin. Results are expressed as fold-changes compared to untreated mice. *P<0.05, **P<0.01 according to Mann-Whitney test.

FIG. 8: Emricasan dampens the M2-polarization of CSF-1-derived macrophages. Human monocytes were differentiated during 5 days with 50 ng/mL CSF-1 and then polarized into MO-macrophages (CSF-1) or M2-macrophages (IL-4) for 24 (mRNA) or 48 hours. Emricasan (3 μM) was added 16h before the polarization. The expression of the indicated mRNA is analyzed by qPCR (mean ±SEM of 6 independent experiments). *P<0.05, **P<0.01 according to a paired student t test (versus M2-macrophages).

### EXAMPLE

Material and Methods

Reagents and Antibodies

Human CSF-1 was purchased from Miltenyi (130-096-493). Emricasan (IDN-6556) was purchased from Euromedex (S7775-5mg). Q-VD-OPh was from Clinisciences (A1901-5 mg). Caspase-8, Caspase-3, Caspase-7 and HSP60 antibodies were purchased from Cell Signaling Technology (catalog numbers were 9746, 9662, 9492 and 12165 respectively). Mouse caspase-8 was from R&D Systems (AF705). HRP-conjugated rabbit anti-goat was purchased from Dako (P0449) and HRP-conjugated goat anti-rabbit was from Cell Signaling (5127). Active recombinant caspase-8 (ALX-201-062) and −3 (ALX-201-059) were from Enzo life sciences.

Human Monocyte Culture and Differentiation

Human peripheral volunteers were obtained from healthy donors with informed consent following the Declaration of Helsinki according to recommendations of an independent scientific review board. The project has been validated by The Etablissement Francais du Sang, the French national agency for blood collection (protocol N°ALM/PLER/FO/001). Blood samples were collected using ethylene diamine tetraacetic acid-containing tubes. Mononucleated cells were first isolated using Ficoll Hypaque (Eurobio, CMSMSL0101). Then, we used the autoMACS® Pro Separator (Miltenyi, France) to perform cell enrichment. An initial positive selection, which included antibody targeting CD14, was used for monocyte enrichment (Miltenyi, 130-050-201). Purified monocytes from human were grown in RPMI 1640 medium with glutamax-I (Life Technologies, 61870044) supplemented with 10% (vol/vol) foetal bovine serum (Life Technologies). Macrophage differentiation was induced by adding into the culture medium 100 ng/mL CSF-1 and was visualized using standard optics (20x/0.35 Ph1) equipped with an AxioCam ERc camera (Zeiss, France). Phase images of the cultures were recorded with the Zen 2 software (Zeiss).

Flow Cytometry

To analyze the macrophagic differentiation of monocytes, the cells were washed with ice-cold phosphate buffered saline (PBS, Life Technologies, 14190169), incubated at 4° C. for 10 min in PBS/bovine serum albumin (BSA 0.5%, Dutscher, 871002) with anti-CD16, anti-CD71 and anti-CD163 or isotype controls (Miltenyi and BD Biosciences, catalog numbers were 130-113-396, 130-097-628 and 551374). Finally, the cells were washed and fixed in 2% paraformaldehyde (EMS, 15710). To perform macrophage polarization, purified monocytes were plated at 0.3×106 per mL in RPMI 1640 medium with glutamax-I supplemented with 10% (vol/vol) fetal bovine serum plus CSF-1 for 5+2 days to differentiate into M0 macrophages. 20 ng/mL IL-4 (Miltenyi, 130-094-117) was added after 5 days of differentiation for two days to polarize into M2-macrophages. To analyze the macrophage polarization, cells were detached using PBS/EDTA/BSA, washed with PBS, and incubated at 4° C. for 10 min in PBS/bovine serum albumin with anti-CD200R (Biolegend, 329308), anti-CD206 (Miltenyi, 130-100-034) and anti-CD163 (Miltenyi, 130-097-628) or isotype controls. Finally, the cells were washed and fixed in 2% paraformaldehyde (EMS, 15710). Fluorescence was measured with a MACSQuant® Analyzer (Miltenyi, Paris, France). To analyze the cell death, cells were washed with ice-cold PBS and incubated at 4° C. for 15 min in a specific buffer (10 mM HEPES, 150 mM NaCl, 5 mM KCl, 1 mM MgCl2, 1 mM CaCl2) with AnnexinV-FITC (Miltenyi, 130-097-928) and DAPI (Sigma-Aldrich, D9542). Fluorescence was measured with a MACSQuant® Analyzer (Miltenyi, Paris, France). To analyze the ability of macrophages to phagocyte bacteria, we used Vybrant® Phagocytosis Assay Kit according manufacture's instruction (ThermoFisher, V-6694). Briefly, macrophages were detached and incubated with fluorescein-labeled E. coli (K-12 strain) for 30 min. Next, cells were washed twice with PBS and resuspended in PBS. Fluorescence, that indicate the internalization of particles, was measured with a MACSQuant® Analyzers (Miltenyi, France). Trypan blue solution was used to quench the fluorescence from particles that were not internalized. To detect caspase activity, we used FITC-DEVD-FMK or FITC-IETD-FMK according to the manufacturer's instruction (Promocell, green caspase-3 or caspase-8 staining kits, PK-CA577-K183 or PK-CA57-188).

Caspase Activity Measurement Assay

After stimulation, cells were lysed for 30 min at 4° C. in lysis buffer (50 mM HEPES pH 8, 150 mM NaCl, 20 mM EDTA, 1 mM PMSF, 10 μg/mL leupeptin, 10 μg/mL aprotinin and 0.2% Triton X-100) and lysates were cleared at 16 000 g for 15 min at 4° C. Each assay (in triplicate) was performed with 10 μg of protein prepared from control or stimulated cells. Briefly, cellular extracts were then incubated in a 96-well plate with 0.2 mM of DEVD-AMC (Caspase-3) or IETD-AMC (Caspase-8) as substrates for various times at 37° C. Caspase activity was measured either following emission at 460 nm (excitation at 390 nm) in the presence or not of 10 μM of DEVD-CHO or IETD-CHO. Enzyme activities were expressed in arbitrary units (A.U.) per min and per mg of proteins. The same protocol was used with 0.25 units of active recombinant caspase-8 (Enzo, ALX-201-062) or -3 (Enzo, ALX-201-059) in each triplicate.

Immunoblot Assays

Cells were lysed for 30 min at 4° C. in lysis buffer [50 mM HEPES pH 7.4, 150 mM NaCl, 20 mM EDTA, PhosphoSTOP (Sigma, 04906837001), complete protease inhibitor mixture (Sigma, 11836153001), 1% Triton X-100 (Sigma, T9284)]. Lysates were centrifuged at 20,000 g (15 min, 4° C.) and supernatants were supplemented with concentrated loading buffer (4× Laemmli buffer). Fifty micrograms of proteins were separated and transferred following standard protocols before analysis with the chemiluminescence detection kit (GE Healthcare, RPN2105).

Whole-Transcriptome RNA-seq

The RNA integrity (RNA Integrity Score≥7.0) was checked on the Agilent 2100 Bioanalyzer (Agilent) and quantity was determined using Qubit (Invitrogen). SureSelect Automated Strand Specific RNA Library Preparation Kit was used according to manufacturer's instructions with the Bravo Platform. Briefly, 50 to 200 ng of total RNA sample was used for poly-A mRNA selection using oligo(dT) beads and subjected to thermal mRNA fragmentation. The fragmented mRNA samples were subjected to cDNA synthesis and were further converted into double stranded DNA using the reagents supplied in the kit, and the resulting dsDNA was used for library preparation. The final libraries were bar-coded, purified, pooled together in equal concentrations and subjected to paired-end sequencing on Novaseq-6000 sequencer (Illumina) at Gustave Roussy.

RNA-Sequencing Analysis

Quantification. Quality of raw FastQ files was assessed with Fastqc v0.11.8 and Fastq-screen v0.13.0. Quality report was gathered with MultiQC v1.8. Abundance estimation was performed with Salmon v0.14.1 using following parameters: —libType A —validateMappings —numBootstraps 60. Salmon index was created using Human Gencode reference annotation release 33 and using following parameters —gencode —keepDuplicates. Differential analysis. Statistical analysis was performed using R 3.6.1. Transcript expression levels were aggregated in gene expression levels using tximport v1.14.0 Bioconductor package. At this step only protein coding genes were considered. We also decided to keep only high quality annotations therefore genes annotated as “automated annotation” in GENCODE were discarded. DESeq2 v1.26.0 method was used to identify differentially expressed genes between groups with an adjusted p-value threshold of 0.05.

Reverse-Transcription and Real-Time Polymerase Chain Reaction

RNA was prepared from 5×106 cells using the RNeasy Mini Kit according to manufacturer's protocol (Qiagen, 74104). Each cDNA sample was prepared using AMV RT and random primers (Promega, M510F and C1181). Real-time polymerase chain reaction (PCR) was performed using the SyBR Green detection protocol (Life Technologies, 4367659). Briefly, 5 ng of total cDNA, 500 nM (each) primers, and 5 μL SyBR Green mixture were used in a total volume of 10 μL. Detection of multiple endogenous controls (ACTB, L32 and UBIQUITIN) were used to normalize the results. Specific forward and reverse primers are accessible upon request.

Animal Models

C57/BL6 female mice (8 weeks-old) were purchased from Charles River Laboratories (L'arbresle, France). Caspase-8 flox/flox mice were kindly provided by Hedrick's laboratory (UCSD) (PMID:16148088) and crossed with LysMCre transgenic mice (PMID: 10621974). Animal genotyping was done by PCR using primers indicated in Table 1, and by immunoblotting.

Lung Fibrosis Model

Procedures were approved by our Institutional Ethical Committee (CEEA 26) and the French Ministry of Research (#9861). Animals were injected intraperitoneally with bleomycin sulfate (0.1 mg/g body weight) once a week during three weeks with or without subcutaneous injection of Emricasan (18 μg/g body weight) (MedChemtronica) twice a day. To quantify the extent of collagen fibers, left lungs were fixed in 4% formaldehyde, paraffin embedded, cut into 4 μm sections, stained with Sirius Red, scanned using a microscopy virtual slide system (Olympus VS120), and analyzed using ImageJ 1.50b software. To quantify airspace number, tissue sections 4-μm stained with Sirius Red were scanned using a NanoZoomer-SQ (Hamamatsu Corporation, Japan). Images of entire lung sections were recorded by means of NDP.view.2 software (Hamamatsu Corporation) and analyzed at ×20 magnification with a pixel size of 0.452 μm. To quantify fibrosis, we used a numerical software program that allows a fully automatic selection of airspaces (alveoli and ducts) from the entire lung sections, without the large bronchi and vessels. Fibrosis severity was indicated by the ratio between the number of airspaces and the total area of parenchymal tissue.

Macrophage Collection and Analysis

Right lungs were digested with the Lung Dissociation kit (Miltenyi Biotec, Somerville, Mass., USA) and filtered before eliminating erythrocytes with ACK to collect nucleated cells. These cells were washed with ice-cold PBS, incubated with Fc block (Murine TruStain FcX, Biolegend, London, UK, 1/50 dilution) for 15 min, incubated with antibodies (Table 1) for 20 minutes at 4° C., washed, and analyzed with a BD LSRFortessa X-20 flow cytometer and FlowJo software v. 10.0.00003. Interstitial macrophages were selected according to their larger size (FSC) and granularity (SSC) as CD45+, GR1−, CD11b high, SiglecF−, IAIE+, CD24− cells, alveolar macrophages as CD45+, GR1−, CD11b low, SiglecF high cells and inflammatory monocytes were selected as CD45+ positive, CD11b high, SiglecF−, IA-IE− cells.

Broncho-Alveolar Lavage Fluid

We collected the broncho-alveolar fluid (BALF) of sacrificed animals by cannulating their trachea and ligating their right lung before slowly delivering 300 μl PBS in the left lung and retrieving the liquid through the cannula, which was repeated twice. BALF was centrifuged at 600 g for 10 minutes at 4° C. before collecting the acellular fraction that was kept at −80° C., up to cytokine analysis. Interleukin-2 (IL-2), IL-5, IL-6, chemokine (C-X-C motif) ligand 1 (CXCL1 or KC), were quantified using Mouse Pro-Inflammatory Panel 1 V-Plex according to the manufacturer's guidelines (MSD), the chemiluminescence signal being measured on a Sector Imager 2400 (MSD). A Milliplex TGFβ1, Single Plex magnetic bead kit (Merck Millipore) and the Bio-Plex200 system (Bio-Rad) were used to measured TGFβ1.

Statistical Analysis

Statistical analysis was performed using a paired Student t test and significance was considered when P values were lower than 0.05. The results are expressed as the mean±SEM. For mouse experiment analyses, investigators were blinded. Data are presented as means±SE. Statistical significance was determined by Mann-Whitney test. All the tests were two-tailed.

Results

Low Concentrations of Emricasan Inhibit CSF-1-Induced Monocyte Differentiation

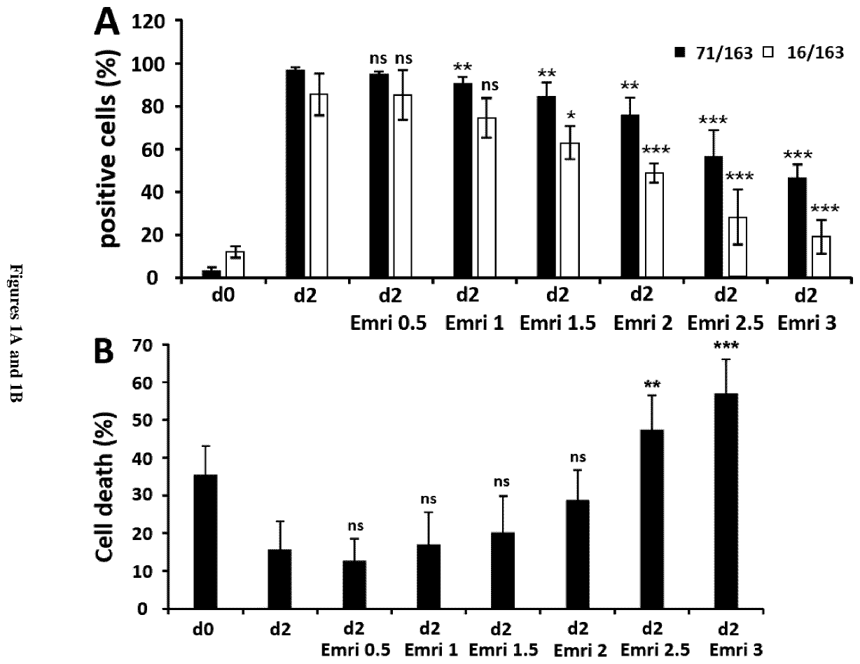

Caspase inhibition using pancaspase inhibitors such as z-VAD-fmk or Q-VD-OPh inhibits CSF-1-induced monocyte differentiation (Jacquel et al., Blood 2009, Sci Reports 2018). We investigated here the effect of increasing concentrations of Emricasan, a pancaspase inhibitor that has recently achieved phase 2 clinical trials in patients suffering liver failure, on human primary monocyte differentiation induced by CSF-1 (FIG. 1). Human primary monocytes treated with CSF-1 for 2 days exhibited a robust increase in the expression of CD71/CD163 and CD16/CD163 antigens, a hallmark of macrophagic differentiation, generating 98% and 92% of double positive cells, respectively, as assessed by flow cytometry (data not shown). Emricasan added at day 0 triggered a dose-dependent inhibition of macrophagic differentiation in the low micromolar range. Quantification of the Emricasan effect in three different donors confirmed a strong inhibitory effect of this pancaspase inhibitor at low micromolar concentrations (1-2 μM) (FIG. 1A).

Induction of differentiation by CSF-1 is known to inhibit the spontaneous apoptosis of monocytes that occurs rapidly in culture in the absence of this cytokine. We thus analyzed the effect of various concentrations of Emricasan on apoptosis induction in differentiating monocytes. CSF-1 reduced apoptotic cell rate three times as shown by annexin V staining at 48 h compared to untreated monocytes (FIG. 1B). Emricasan failed to induced significant loss of Annexin V staining at low concentrations, but slightly increased DAPI staining at higher concentrations (3 μM). In conclusion, Emricasan did not induce apoptosis in undifferentiated monocytes and low concentrations of Emricasan (up to 2 μM) were not toxic for primary human monocytes, indicating that Emricasan can be used beneficially in place of other pancaspase inhibitors.

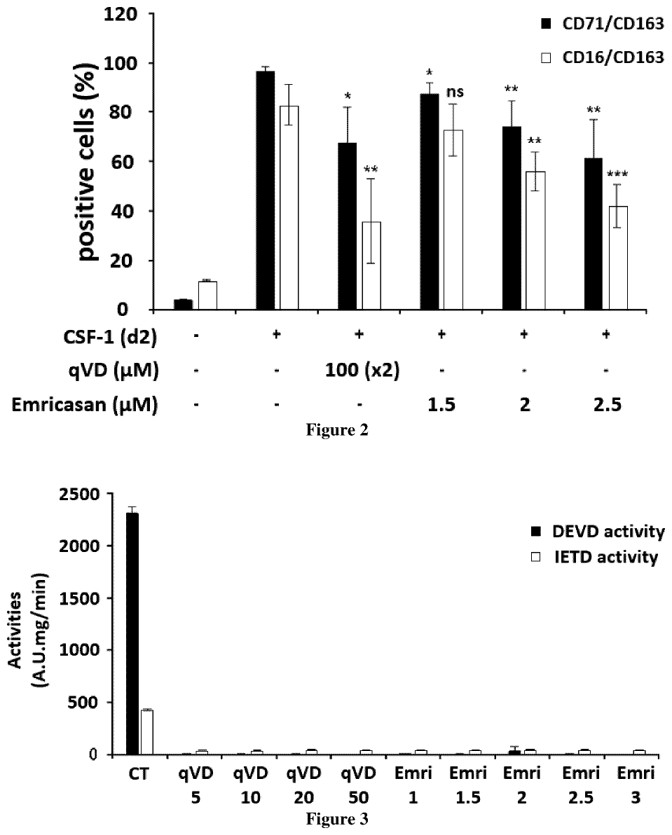

We therefore investigated in comparison with Emricasan the inhibitory effect of Q-VD-OPh, a pancaspase inhibitor widely used in the literature to block apoptotic caspases, on CSF-1-induced monocyte differentiation. Monocytes treated with CSF-1 for 2 days in the absence of Q-VD-OPh, exhibited an increased expression of CD71/CD163 and CD16/CD163 antigens generating 97% and 78% of double positive cells, respectively, as assessed by flow cytometry (data not shown). Q-VD-OPh added at day 0 triggered a dose-dependent inhibition of macrophagic differentiation in the 75-125 μM range that was however weak compared to the effect of Emricasan (data not shown). When Q-VD-OPh was added twice, i.e at day 0 and day 1 a much robust inhibition of monocyte differentiation was achieved, that was however weaker than the one obtained with Emricasan used at a single and lower dose (data not shown). Quantification of the Q-VD-OPh effect on several different donors confirmed a significant inhibitory effect of this pancaspase inhibitor at 100-125 μM, but only when added twice (data not shown). We next directly analyzed on the same experiment the ability of Q-VD-OPh added twice (at days 0 and 1) and Emricasan added only one time to impair CSF-1-mediated monocyte differentiation (data not shown). As expected, monocytes treated with CSF-1 for 2 days exhibited a robust increase in the expression of CD71/CD163 and CD16/CD163 antigens, generating 95% and 74% of double positive cells, respectively, as assessed by flow cytometry. Emricasan in the 1.5-2.5 μM range efficiently inhibited monocyte differentiation while 2 successive treatments with100 μM Q-VD-OPh were necessary to achieve an identical inhibition, further demonstrating the superiority of Emricasan towards Q-VD-OPh. Quantification of these results on 3 different donors confirmed the higher potency of Emricasan versus Q-VD-OPh to inhibit CSF-1-induced monocyte differentiation into macrophages (FIG. 2). All together these data indicate that Emricasan is a highly potent inhibitor of caspases during differentiation of human monocytes into macrophages and can be used beneficially instead of the less active and specific compounds Q-VD-OPh or z-VAD-fmk.

Both Q-VD-OPh and Emricasan are Potent Inhibitors of Apoptosis in Untreated Monocytes

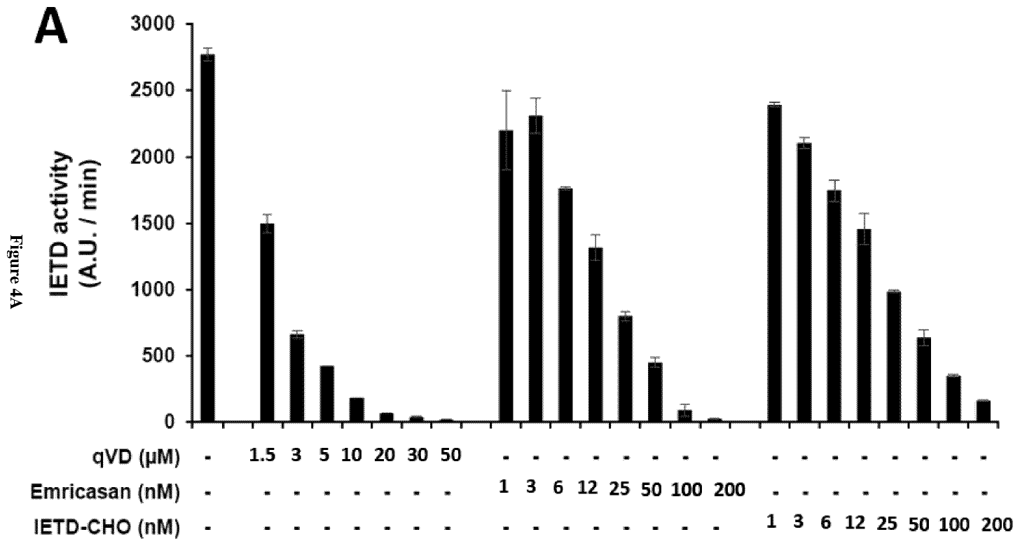

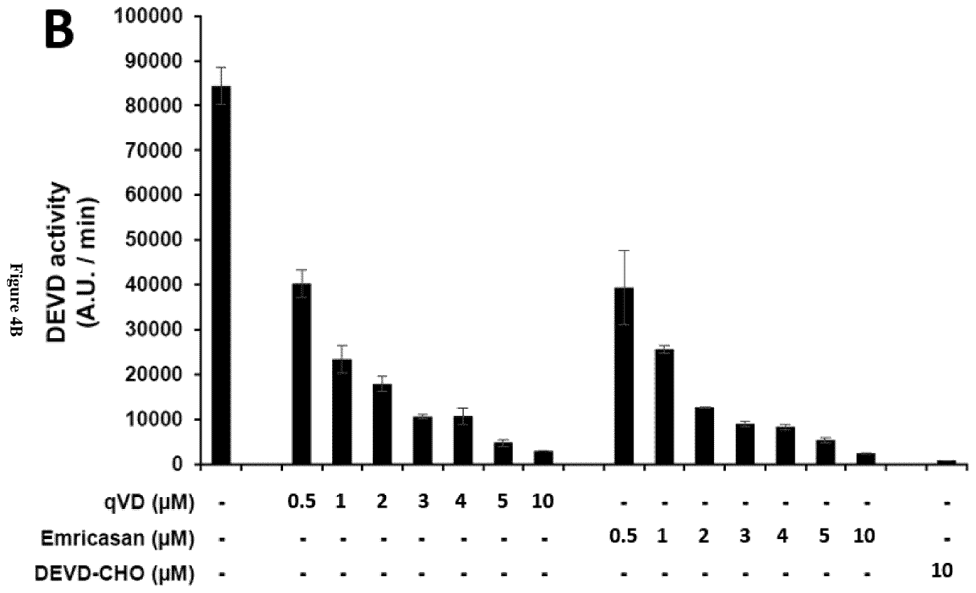

When cultured in the absence of CSF-1, human monocytes rapidly underwent apoptosis as assessed by Annexin V/DAPI staining (FIG. 1B). Human freshly isolated monocytes were left untreated or treated with different concentrations of Q-VD-OPh or Emricasan for 24 h. In the absence of pancaspase inhibitors, 51% of monocytes exhibited increased Annexin V staining at 24 h, indicative of apoptotic cell death induction. Concentrations of Q-VD-OPh as low as 5 μM were sufficient to abrogate Annexin V staining after 24 h in culture without CSF-1 indicating that Q-VD-OPh is much more efficient to block caspase activation induced during apoptosis than during CSF-1-mediated differentiation. A single concentration of Emricasan (2 μM) was sufficient to obtain the same effect (data not shown). We confirmed by Western Blot experiments that both inhibitors abrogated the cleavage of the zymogens of caspases 8, 3 and 7 in their active 17-20 kDa fragments (data not shown). Finally, we also verified that all the concentrations of Q-VD-OPh and Emricasan efficiently inhibited caspase 3 activity in untreated monocytes, using Ac-DEVD-AMC as substrate (FIG. 3). Therefore it appears that Q-VD-OPh is a potent inhibitor of apoptotic caspases but conversely to Emricasan, a weaker inhibitor of the activation of caspases that specifically occurred and are essential for proper monocyte differentiation. As a whole these findings show that Emricasan is more active on those caspase activities that trigger differentiation of monocytes.

Effect of Caspase Inhibitors on Recombinant Caspases-8 and -3

To investigate further the differential effect of Q-VD-OPh and Emricasan on caspase activities and monocyte differentiation, we performed dose-response curves for both inhibitors on recombinant caspase-8 and -3 activities in vitro. Caspase-8 was assessed using Ac-IETD-AMC as substrate. Q-VD-OPh abrogated caspase-8 activity at 50 μM, with an IC50 around 1 μM, whereas Emricasan fully inhibited caspase-8 activity at 0.2 μM and exhibited an IC50 of only 0.012 μM, that was in the range of Ac-IETD-CHO, a highly potent caspase-8 inhibitor (IC50=0.015 μM) (FIG. 4A). The same experiment was reproduced using recombinant caspase-3 and Ac-DEVD-AMC as substrate (FIG. 4B). Importantly, the dose-response curve for Q-VD-OPh and Emricasan inhibition of caspase-3 were perfectly stackable (maximal inhibition at 10 μM and IC50 in the 0.5 μM range), indicating that both inhibitors were equally efficient to inhibit recombinant caspase-3 in vitro. In conclusion, the better efficiency of Emricasan to inhibit CSF-1-induced human monocyte differentiation in vitro and ex vivo likely relies on its ability to abrogate caspase-8 activity which is crucial for this process.

Emricasan Efficiently Inhibits Caspase 8 Activity in Cellulo in Differentiating Monocytes

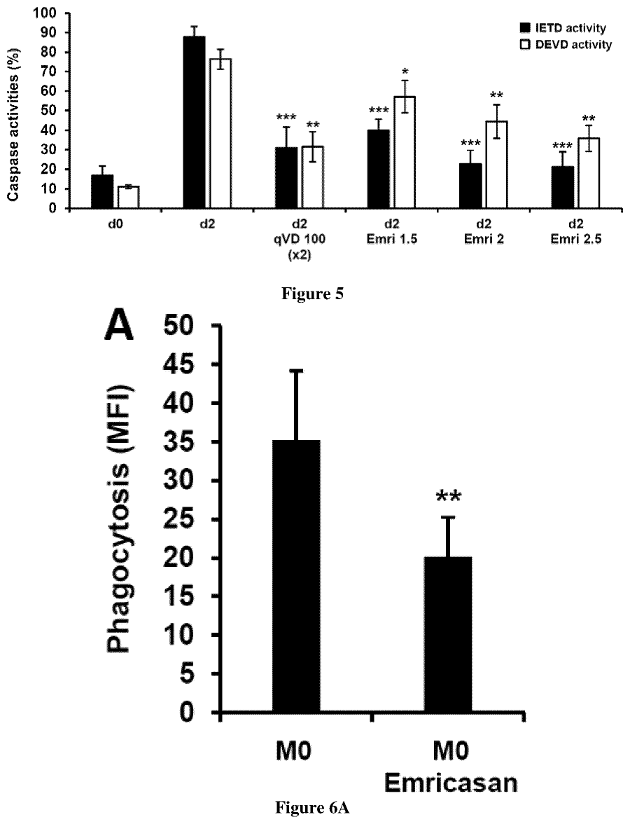

We have shown previously that the superiority of Emricasan compared with Q-VD-OPh relies on its better efficiency towards caspase-8 in vitro using a recombinant caspase. To assess caspase-8 activity in cellulo, human primary monocytes were incubated with or without CSF-1 in either the presence of different concentrations of Emricasan or a maximal concentration of Q-VD-OPh (100 μM, 2 times). After 2 days, caspase-8 activity was assessed by flow cytometry using Ac-IETD-FITC as substrate (data not shown). 88% of differentiated monocytes exhibited high caspase-8 staining, indicative of caspase-8 activation (data not shown). Q-VD-OPh added twice at 100 μM induced a strong inhibition of caspase-8 activity, whereas Emricasan abrogated caspase-8 activity at the single dose of 2 μM, in agreement with its effect on monocyte differentiation (FIGS. 1A and 1B and FIG. 2). Quantification of caspase-8 activity in several experiments confirmed a very strong inhibition of the percentage of cells expressing active caspase-8 (FIG. 5). A potent inhibition of caspase -3 activity in cellulo was observed in identical conditions (FIG. 5).

We also checked in parallel that the different caspase inhibitors blocked CSF-1-mediated monocyte differentiation (data not shown). Finally, we verified using western blot experiments the cleavage of caspases-3 and -7 in their differentiation-like characteristic fragments of 26 and 30 kDa in monocytes treated 2 days with CSF-1 (data not shown). Importantly, we established that Q-VD-OPh and Emricasan impaired the cleavage of effector caspases-3 and -7 at their specific differentiation cleavage site.

Emricasan Impedes Macrophage Polarization Ex Vivo

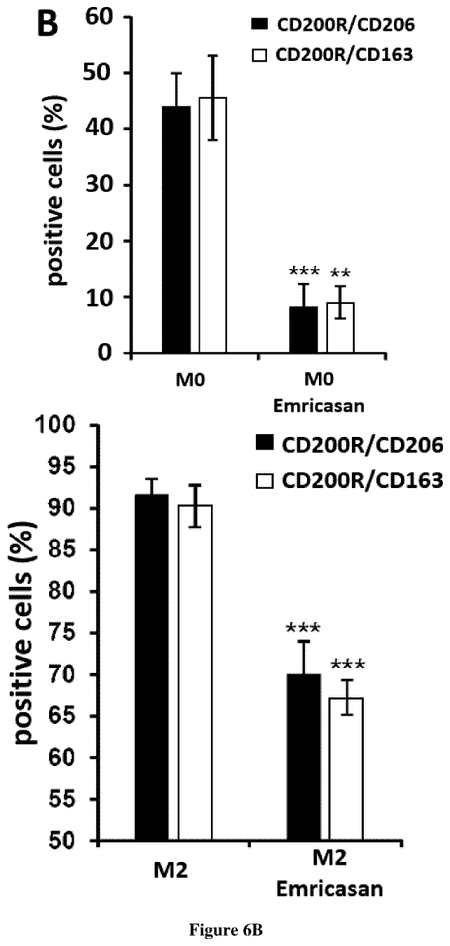

Although caspases are necessary for the differentiation of monocytes into macrophages induced by CSF-1, their role in the polarization of macrophages into the M1 or M2 phenotype has not been explored so far. To investigate a possible implication of caspases during these processes, primary human monocytes were firstly incubated 5 days with CSF-1 to induce macrophagic differentiation, and next treated with IL-4 during 2 days to induce polarization towards M2 phenotype. Emricasan dampened M2-like polarization of M0 macrophages induced by IL4 (FIGS. 6A and 6B) and at the same time induced M1-like markers.

Emricasan Impedes Monocyte-Derived Cell Changes Induced by IL-4

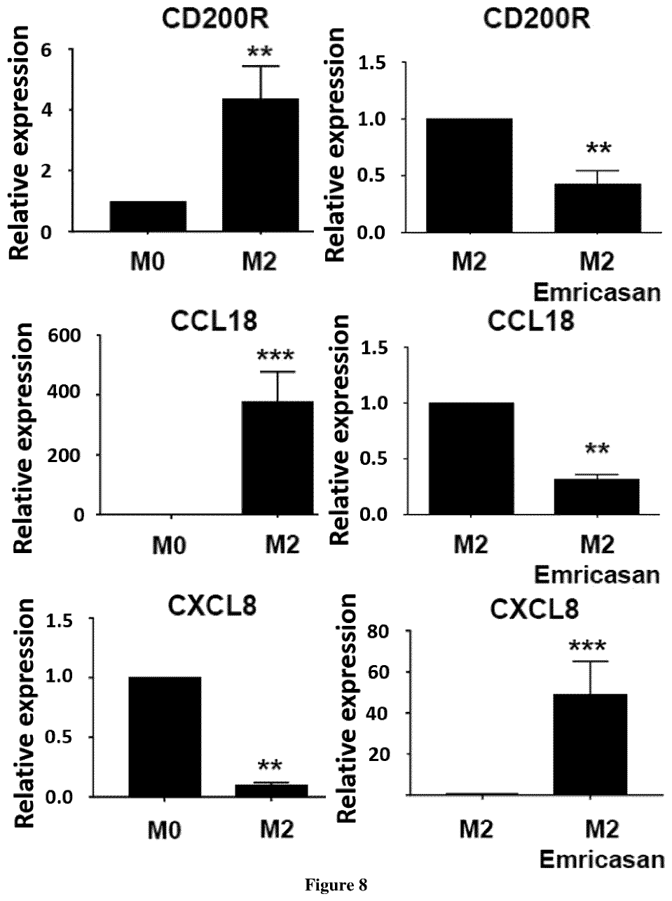

In IL4-polarized macrophages, Emricasan inhibited the generation of CD200R+/CD206+ and CD200R+/CD163+ double-positive cells (FIG. 6B lower panel). To confirm the inhibitory effect of Emricasan on IL4-mediated M2 polarization, we performed RNAseq analysis on macrophages from three different donors. We found that IL-4 both induces anti-inflammatory and represses pro-inflammatory markers, an effect that was counteracted by Emricasan (data not shown). This mirror effect was confirmed by analyzing the mRNA level of CD200R and CCL18, two well-known anti-inflammatory molecules, and CXCL8, a proinflammatory one. Indeed, CD200R and CCL18 expressions were increased by IL-4, an effect counteracted by Emricasan, while CXCL8 level was diminished by IL-4 but upregulated when IL-4 was combined with Emricasan (FIG. 8). In conclusion, caspase inhibition by Emricasan prevented the up-regulation of anti-inflammatory actors to the benefit of pro-inflammatory molecules, thus orienting polarization of human macrophages towards a pro-inflammatory phenotype.

Caspase-8 Inhibition Prevents Bleomycin-Induced Lung Fibrosis in Mice

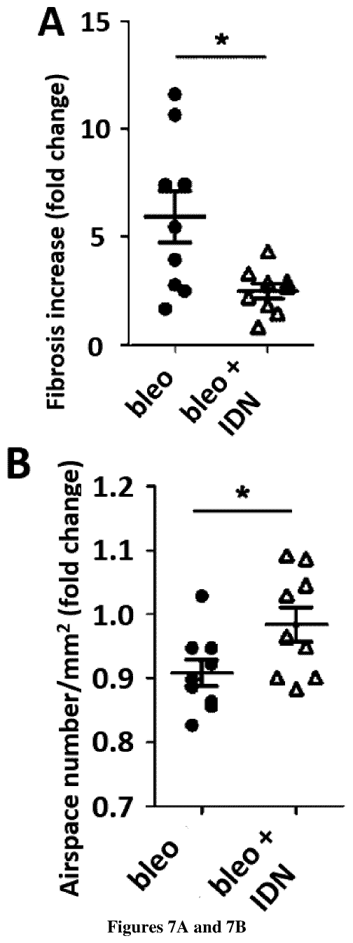

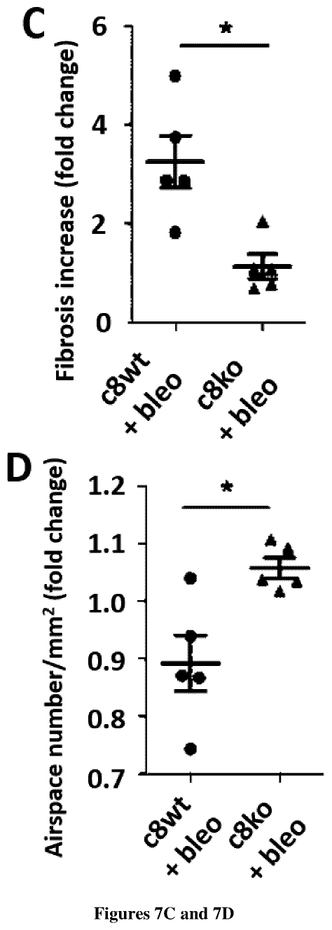

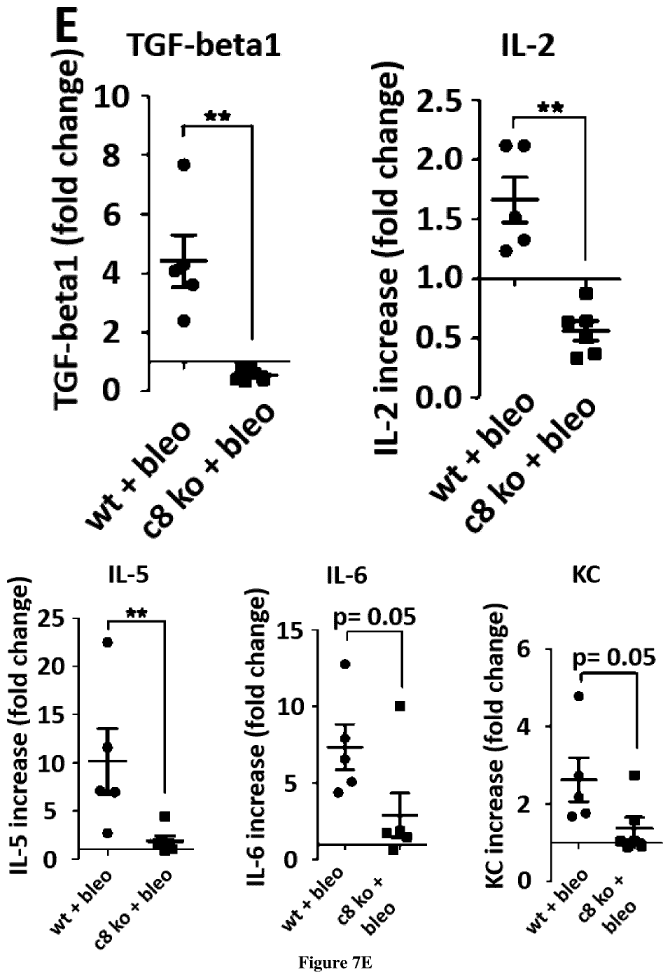

Emricasan has been shown to improve liver functions in patients suffering liver diseases in several recent studies (Frenette C T, Clin Gastroenterol Hepatol. 2019 Mar; 17(4):774-783 ; Barreyro F J, Liver Int. 2015 Mar; 35(3):953-66 ; Baskin-Bey E S, Am J Transplant. 2007 Jan; 7(1):218-25). We investigate whether this could be also the case in bleomycin-induced pulmonary fibrosis in mice, a disease associated with M2-macrophage infiltration. Weekly intraperitoneal injection of bleomycin sulfate (0.1 mg/g body weight) to 2-month old mice generates a lung fibrosis that, after three weeks, can be visualized by Sirius Red staining of collagen fibers (data not shown) and quantified using ImageJ 1.50b software (FIG. 7A). Complete obliteration of alveoli, which is a key feature of pulmonary fibrosis, provokes a decrease in airspace number that can also be quantified (FIG. 7B). Subcutaneous injection of Emricasan twice a day (18 μg/g body weight) for three weeks dramatically decreases lung fibrosis intensity (data not shown), as verified by quantifying Sirius Red staining intensity (FIG. 7A) and air space (FIG. 7B). To further explore the role of caspases in bleomycin-induced lung fibrosis, we generated mice with LysM promoter guided, Cre recombinase-induced deletion of caspase-8 (Caspase-8-/−). Genotyping of the models validated both the presence of the foxed alleles in mouse tail DNA and the appropriate deletion of targeted alleles in macrophages (data not shown). LysM-Cre driven caspase-8 gene deletion protected the animals from bleomycin induced lung fibrosis (data not shown), as indicated by a decreased network of collagen fibers (FIG. 7C) and a lower restriction of airspace (FIG. 7D). Analysis of cytokines in the broncho-alveolar fluid (BALF) collected from bleomycin treated animals revealed a decreased level of TGFβ1, IL-2, IL-5, IL-6 and KC in the BALF of Caspase-8-/− mice (FIG. 7E).

Conclusion:

In conclusion, we first established that compared to Q-VD-OPh, a classically and widely used pancaspase inhibitor, Emricasan durably impairs CSF-1-induced monocyte differentiation and this at very low micromolar range and therefore represents an excellent alternative to other pancaspase inhibitors. Moreover, we confirmed in vitro using recombinant caspases the much more greater efficiency of Emricasan on caspase-8. By contrast, we found no difference in the ability of Q-VD-OPh and Emricasan to inhibit caspase-3 activity in vitro and to impair apoptosis of human monocytes ex vivo suggesting an equivalent effect of both inhibitors on caspase-3. More precisely, we found that Emricasan was 100 times more potent on caspase-8 activity and monocyte differentiation than Q-VD-OPh. These original results demonstrate that Emricasan can be used to block the specific activation of caspases observed during CSF-1-mediated differentiation of monocytes into macrophages. Indeed, in response to CSF-1, a caspases activation cascade is initiated by the cleavage and the activation of caspase-8 in an original multimolecular complex composed of FADD, FLIP, RIP1 and caspase-8. Importantly, and that's what makes it so original, there is no cell death receptor within this platform. Caspase-8 activation secondly triggers the cleavage and activation of caspase-3 that ultimately cleaves several protein substrates, among which some such as NPM1 (nucleophosmin 1) are thought to play a role in the differentiation process. Interestingly, the cleavage site of effector caspases, i.e. caspase-3 and caspase-7, during monocyte differentiation are completely different from the one are cleaved when monocytes underwent spontaneous apoptosis in the absence of CSF-1 (data not shown). This distinct mode of caspases cleavage, and accordingly their specific activation, may explain the differences observed in the sensitivity to various caspase inhibitors observed during differentiation of monocytes. Once differentiated, macrophages play an important role in tissue development, inflammation, anti-pathogenic defense, homeostasis and cancer and their major functions are phagocytosis, antigen presenting and cytokine production. In activated immune responses, macrophages are a heterogenous population, exerting a combination of pro-inflammatory (M1-macrophages) and anti-inflammatory (M2-macrophages) functions. Deciphering the process of macrophage polarization, recruitment, and functions may provide insights for the development of new therapies to manipulate the balance of M1/M2 phenotype, number, and distribution of macrophage, in order to enhance anti-microbial defense or dampen detrimental inflammation. In this study, we demonstrated that caspases targeting using Emricasan, a clinically available pan-caspase inhibitor, may be a promising approach to evaluate the ability of Emricasan to modify the M2 polarization of CSF-1-induced macrophages.

### REFERENCES

Throughout this application, various references describe the state of the art to which this invention pertains. The disclosures of these references are hereby incorporated by reference into the present disclosure.

1. A method of polarizing a macrophages, comprising
contacting the macrophage with a caspase 8 inhibitor.

2. The method according to claim 1 wherein the macrophage is a type 2 macrophage.

3. The method according to claim 1 wherein the macrophage is a type 1 macrophage.

4. The method according to claim 1, wherein said caspase 8 inhibitor is Emricasan.

5. A method of treating a macrophage related diseases in a subject in need thereof, comprising
administering to the subject a therapeutically effective amount of a caspase 8 inhibitor.

6. The method according to claim 5, wherein the macrophage related disease is selected from the group consisting: a solid cancer, a fibrotic diseases, hepatic fibrosis, systemic sclerosis, allergy, asthma, atherosclerosis and Alzheimer's disease.

7. The method according to claim 6, wherein the fibrotic disease is lung fibrosis.

8. The method according to claim 6 wherein the solid cancer is selected from the group consisting of: adrenal cortical cancer, anal cancer, bile duct cancer, bladder cancer, bone cancer, brain and central nervous system cancer, breast cancer, cervical cancer, colorectal cancer, endometrial cancer, esophagus cancer, gallbladder cancer, gastrointestinal carcinoid tumors, Kaposi's sarcoma, kidney cancer, laryngeal and hypopharyngeal cancer, liver cancer, lung cancer, mesothelioma, plasmacytoma, nasal cavity and paranasal sinus cancer, nasopharyngeal cancer, neuroblastoma, oral cavity and oropharyngeal cancer, ovarian cancer, pancreatic cancer, penile cancer, pituitary cancer, prostate cancer, retinoblastoma, rhabdomyosarcoma, salivary gland cancer, skin cancer, stomach cancer, testicular cancer, thymus cancer, thyroid cancer, vaginal cancer, vulvar cancer, and uterine cancer.

9. The method according to claim 5, wherein the caspase 8 inhibitor is administered in combination with a classical treatment.

10. (canceled)

11. The method according to claim 9, wherein the classical treatment is administration of a natural or synthetic compound, immunotherapy, chemotherapy or radiotherapy.

12. A pharmaceutical composition comprising a caspase 8 inhibitor.

13. The pharmaceutical composition according to claim 12, wherein the caspase 8 inhibitor is Emricasan.

14. (canceled)

15. The method of claim 8, wherein
the bile duct cancer is peripheral cancer, distal bile duct cancer, or intrahepatic bile duct cancer;
the bone cancer is osteoblastoma, osteochondroma, hemangioma, chondromyxoid fibroma, osteosarcoma, chondrosarcoma, fibrosarcoma, malignant fibrous histiocytoma, giant cell tumor of the bone, chordoma, or multiple myeloma;
the brain and central nervous system cancer is meningioma, astrocytoma, oligodendroglioma, ependymoma, glioma, medulloblastoma, ganglioglioma, Schwannoma, germinoma or craniopharyngioma;
the breast cancer is ductal carcinoma in situ, infiltrating ductal carcinoma, infiltrating lobular carcinoma, lobular carcinoma in situ or gynecomastia;
the endometrial cancer is endometrial adenocarcinoma, adenocanthoma, papillary serous adenocarcinoma, or clear cellcarcinoma;
the gallbladder cancer is mucinous adenocarcinoma or small cell carcinoma;
the gastrointestinal carcinoid tumor is a choriocarcinoma or a chorioadenoma destruens carcinoma;
the kidney cancer is renal cell cancer;
the liver cancer is hemangioma, hepatic adenoma, focal nodular hyperplasia, or hepatocellular carcinoma;
the lung cancer is small cell lung cancer or non-small cell lung cancer;
the paranasal sinus cancer is esthesioneuroblastoma or midline granuloma;
the rhabdomyosarcoma is embryonal rhabdomyosarcoma, alveolar rhabdomyosarcoma or pleomorphic rhabdomyosarcoma;
the skin cancer is melanoma or nonmelanoma skin cancer;
the testicular cancer is seminoma or nonseminoma germ cell cancer;
the thyroid cancer is follicular carcinoma, anaplastic carcinoma, poorly differentiated carcinoma or medullary thyroid carcinoma; and/or
the uterine cancer is uterine leiomyosarcoma.
